(19) United States
(12) Patent Application Publication
(10) Pub. No.: US 2023/0236179 A1
(43) Pub. Date: Jul. 27, 2023

(54) Single Molecule Assays for Ultrasensitive Detection of Biomolecules

(71) Applicant: The Brigham and Women`s Hospital, Inc., Boston, MA (US)

(72) Inventors: David R. Walt, Boston, MA (US); Connie Wu, Boston, MA (US); Adam M. Maley, Peoria, IL (US)

(21) Appl. No.: 18/012,111

(22) PCT Filed: Jun. 22, 2021
(86) PCT No.: PCT/US2021/038463

§ 371 Date: Dec. 21, 2022

Related U.S. Application Data
(60) Provisional application No. 63/076,833, filed on Sep. 10, 2020.
(60) Provisional application No. 63/042,596, filed on Jun. 23, 2020.

(51) Int. Cl. G01N 33/543; G01N 33/53; G01N 33/68

(52) U.S. Cl. CPC G01N 33/54326; G01N 33/5308; G01N 33/6869; G01N 2470/04

(57) ABSTRACT

Provided herein are assays that provide digital measurement methods to detect proteins and other biomolecules, e.g., at low- to mid-attomolar concentrations.

### CLAIM OF PRIORITY

This application claims the benefit of U.S. Provisional Patent Application Ser. Nos. 63/042,596, filed on Jun. 23, 2020, and 63/076,833, filed on Sep. 10, 2020. The entire contents of the foregoing are hereby incorporated by reference.

### TECHNICAL FIELD

Provided herein are improved single molecule assays that provide digital measurement methods to detect proteins and other biomolecules at low- to mid-attomolar concentrations.

### BACKGROUND

The ability to accurately measure extremely low levels of biomolecules, such as proteins, nucleic acids, and metabolites, is essential for a wide range of clinical and environmental applications, including disease diagnostics, drug discovery, pathogen detection in food, environmental toxin detection, and bioprocess control. Ultrasensitive measurement techniques are especially critical in clinical diagnostics, as many potential biomarkers exist in accessible biofluids at levels well below the detection limits of current laboratory methods.¹ Digital measurement methods, such as digital enzyme-linked immunosorbent assay (ELISA), have vastly improved measurement sensitivities by up to 1000-fold over traditionally used analytical techniques such as conventional ELISA.²⁻⁵ However, the sensitivities of digital measurement techniques remain insufficient for many diagnostic applications, particularly for measuring disease-related proteins. For instance, while several protein biomarkers for neurological disorders have been shown to be upregulated in cerebrospinal fluid, highly invasive lumbar punctures are required for these measurements, thus making it impractical to screen individuals for early disease detection.⁶⁻⁹ As only a small fraction of brain-derived proteins passes through the blood-brain barrier into circulation, highly sensitive techniques that can detect and identify rare protein biomarkers through a simple blood test are crucial for addressing this unmet diagnostic need.¹⁰⁻¹² Improving analytical sensitivity is also a major challenge in other diseases for which rapid point-of-care (POC) diagnosis is essential for effective medical intervention but where easily accessible biofluids, such as saliva or urine, are required. These biofluids contain only a minimal serumnal component, necessitating ultrasensitive techniques for protein biomarker detection.

One main barrier towards increasing sensitivity in digital ELISA is low sampling efficiency. While digital ELISA methods utilize single molecule counting to improve measurement sensitivity, low sampling efficiencies limit the number of target molecules that are counted. At very low target concentrations, the Poisson noise from counting single events, \sqrt{N}, where N is the number of counted molecules, contributes significantly to measurement error. As an example, at a sampling efficiency of 5%, only 30 out of the 600 target molecules in 100 μL of a 10 aM sample will be counted, assuming perfect capture efficiencies. The theoretical Poisson noise-associated coefficient of variation (CV), \sqrt{N}/N, is 18% at this low sampling efficiency and in reality much higher when accounting for capture efficiencies well below 100% and experimental error. This high measurement uncertainty therefore poses a major limitation for detecting rare molecules. Increasing sampling efficiencies to count more target molecules can thus greatly improve measurement precision and sensitivity but remains a challenge in digital ELISA.

Existing digital ELISA approaches utilize microwells or water-in-oil droplets to isolate individual beads carrying single target protein molecules.^{2.5, 13-15} The current state of the art for digital ELISA is Single Molecule Arrays (Simoa), which captures single target molecules on antibody-coated paramagnetic beads and isolates individual beads into femtoliter-sized microwells for single molecule counting.² A large excess number of beads over the number of target molecules in the sample is used to ensure digital measurements, where each bead has either zero or one captured target molecule and follows the Poisson distribution. Each captured molecule is labeled with a biotinylated detector antibody to form an immunocomplex sandwich, which is then labeled with the enzyme conjugate streptavidin-β-galactosidase (SβG). The beads are subsequently loaded, along with fluorogenic enzyme substrate into the microwells, each of which can fit at most one bead. Upon sealing of the microwells with oil, a high local concentration of fluorescent product is catalytically generated in each well that contains a bead carrying an SβG molecule. Thus, the number of target molecules is measured by counting “on” and “off” wells.

While Simoa can achieve sub-femtomolar limits of detection and is the current gold standard for ultrasensitive protein detection, its sensitivity is limited by low sampling efficiencies. Only about 5% of the total number of beads can be loaded by gravity into the microwells and analyzed.¹⁶ While an external magnetic force is utilized for bead loading in the most recently developed Simoa instrument, the HD-X Analyzer, the percentage of analyzed beads remains around 5%. Other methods to improve bead loading have also been explored, including electric field-directed bead loading, hydrophilic-in-hydrophobic microwell arrays, and digital microfluidics.^{14,15, 17-19} While these methods have increased bead loading efficiencies, demonstrations of their improvements in digital immunoassay sensitivities remain limited. Furthermore, complex fabrication methods and workflows limit the use of these approaches in POC applications. Another strategy for improving sampling efficiency in digital bioassays is bead encapsulation in water-in-oil droplets. Digital droplet-based immunoassays have been demonstrated with up to 60% bead loading efficiencies and have shown equal or improved sensitivities of up to an order of magnitude higher than that of the current Simoa technology.^{5,13} While droplet microfluidic systems are well established for diverse applications, the need for highly controlled, high-throughput droplet generation introduces additional fabrication and processing steps that introduce more complexity when integrating into POC systems. Furthermore, as a significant fraction of droplets do not contain beads but must still be imaged, improving imaging throughput remains another challenge towards POC implementation.

### SUMMARY

Measurements of very low levels of biomolecules, including proteins and nucleic acids, remain a critical challenge in many clinical diagnostic applications due to insufficient sensitivity. While digital measurement methods such as Single Molecule Arrays (Simoa), or digital ELISA, have made significant advances in sensitivity, there are still many potential disease biomarkers that exist in accessible biofluids at levels below the detection limits of these techniques. Described herein are sensitive digital ELISA platforms that address the abovementioned challenges. The vastly simplified readout process and improved cost-effectiveness of the present methods, which in some embodiments require only a microscope slide for bead loading and a simple optical setup for signal readout, can facilitate potential integration into a POC system. The present platform can achieve attomolar limits of detection, with an up to 25-fold increase in sensitivity over the current (e.g., Simoa and digital ELISA) technology. As a proof of concept, we demonstrated the ability of the present methods to measure previously undetectable levels of Brachyury, a tissue biomarker for chordoma, a rare form of bone cancer, in plasma. The enhanced sensitivity and simplicity of the present methods thus provide a platform for biomarker discovery and POC diagnostic development.

Another exemplary method uses a gel to encapsulate the beads in a monolayer to allow for imaging to be done without the beads moving; all of the reactions are done in solution, followed by gel polymerization around the beads and imaging.

Thus, provided herein are methods for detecting a biomolecule in a sample. The methods include providing a solution comprising the sample; contacting the solution with a plurality of beads comprising a capture moiety that binds to the biomolecule, under conditions and for a time sufficient for biomolecules in the sample to bind to the capture moiety; contacting the solution with a binding moiety (e.g., subsequently or simultaneously with the capture moieties) that binds to the biomolecule and allows for generation of an on-bead non-diffusible detectable signal sufficient to allow detection of each bead carrying a target molecule, and then generating the amplified signal; immobilizing the beads, optionally in a monolayer; and detecting the signal.

In some embodiments, immobilizing the beads comprises dropcasting the solution comprising the beads onto a slide, or catalyzing gelation of the solution.

In some embodiments, the methods include contacting the solution with a signal amplification moiety that binds to the binding moiety.

In some embodiments, the signal amplification moiety comprises an enzyme or branched DNA.

In some embodiments, detecting the signal comprises imaging the beads to detect a fluorescent or other signal. In some embodiments, the beads are immobilized in a monolayer and a single z-section imaging can be used; in embodiments where the beads are not in a monolayer, the methods can include imaging different z sections.

In some embodiments, the methods include determining a number and/or percentage of beads that comprise bead-biomolecule complexes.

In some embodiments, the bead comprises a polymer, metal, metal-oxide, semiconductor, and/or semiconductor oxide.

In some embodiments, the detectable signal is generated by rolling circle amplification followed by hybridization with a complementary fluorescently labeled DNA probe; Tyramide Signal Amplification (TSA); hybridization chain reaction; Enzyme-catalyzed proximity labeling (PL) polymerization; Polymerization-based signal amplification; or Magnetic Bead—Quantum Dot Immunoassays.

In some embodiments, the detectable signal is generated by a pre-amplified signal, e.g., a labeled polymer or nanoparticle.

In some embodiments, the beads are dropcast onto a surface and allowed to dry, e.g., to form a film, or the solution is applied to or in contact with a surface and gelation is catalyzed, before the signal is detected.

In some embodiments, the surface is a slide, chip, or flowcell.

In some embodiments, the catalyzing gelation of the solution comprises mixing fibrinogen and/or thrombin; fibrin; cellulose; collagen; gelatin; agarose; hyaluronic acid; polyhydroxyethylmethacrylate (poly(HEMA)); polyethylene glycol (PEG); or acrylamide into the solution.

In some embodiments, the solution comprises a polymer selected from fibrinogen and/or thrombin; fibrin; cellulose; collagen; gelatin; agarose; hyaluronic acid; polyhydroxyethylmethacrylate (poly(HEMA)); polyethylene glycol (PEG); or acrylamide; and the method comprises catalyzing gelation of into polymer.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Schematic of exemplary dropcast single molecule assays. Upon formation of single immunocomplex sandwiches on antibody-coated paramagnetic beads and labeling with a streptavidin-DNA conjugate, rolling circle amplification (RCA) is performed on the beads to generate a long concatemer attached to each immunocomplex. Fluorescently labeled DNA probes are hybridized to the concatemer during RCA to produce a localized fluorescent signal on beads carrying a full immunocomplex sandwich. After RCA, the beads are concentrated, dropcast onto a microscope slide, and allowed to dry to form a monolayer film. Single target molecules are counted by fluorescent imaging of the dropcast film and counting “on” and “off” beads.

FIGS. 2A-G. Imaging of films. (A) Representative photograph and (B) brightfield image of dropcast bead films on a microscope slide. Approximately 2000-2500 beads are analyzed in each frame. Scale bar=100 μm. (C-E) Representative images of “on” and “off” dye-encoded beads in dropcast film: bead fluorescence (488 nm; C), ATTO 647N probe (647 nm; D), and merged (E). Grey arrows indicate “on” beads. Scale bar=10 μm. (F-G) Representative histograms of the maximum fluorescence intensity values (subtracted from background fluorescence intensity in the image) on each bead for 0 fM IL-1β(F) and 10 fM IL-1β(G) samples. A normal distribution was fitted to the fluorescence intensity values, and the cutoff for an “on” versus “off” bead was determined as five standard deviations above the mean.

FIGS. 3A-F. Comparisons of the present methods and conventional Simoa assay sensitivities. (A) the present methods and (B) conventional Simoa calibration curves for human IL-10. Dashed lines indicate the calculated limits of detection (LODs). (C) Comparison of signal to background ratios between the present methods and conventional Simoa across the IL-10 calibration curve range. (D) the present methods and (E) conventional Simoa calibration curves for human IL-1β. (F) Comparison of signal to background ratios between the present methods and conventional Simoa across the IL-1(3 calibration curve range.

FIGS. 4A-B. Effect of sampling efficiency on measurement precision and sensitivity. (A) Measurement CVs of the background signal and (B) calculated LODs for randomly selected subsets of beads imaged using the present methods calibration curve for IL-10. The percentage of beads analyzed represents the percentage of total assay beads. Each point represents the average of four different randomly selected subsets of beads.

FIGS. 5A-F. Measurements of Brachyury in plasma. (A) the present methods and (B) conventional Simoa calibration curves for human Brachyury. Dashed lines indicate the calculated limits of detection (LODs). (C) Comparison of signal to background ratios between the present methods and conventional Simoa across the calibration curve range. (D-E) Average enzyme per bead (AEB) and average molecule per bead (AMB) values measured by conventional Simoa and the present methods, respectively, in chordoma patient plasma samples (D) and commercial plasma and serum samples (E). Brown lines indicate LODs of the assays. (F) Measured concentrations in chordoma, chondrosarcoma, and commercial plasma and serum samples using the present methods and conventional Simoa. Measurements below the LOD were assigned a value of zero.

FIGS. 6A-B. Validation of human IL-10 dSimoa assay in pooled human saliva. (A) Recoveries of spiked recombinant human IL-10 protein in pooled human saliva diluted four-fold. (B) Measured IL-10 concentrations in serially diluted samples of pooled human saliva.

FIGS. 7A-B. Validation of human Brachyury dSimoa assay in human plasma and serum. (A) Recoveries of spiked recombinant human Brachyury protein in individual commercial human serum samples and pooled human plasma diluted eight-fold. (B) Measured Brachyury concentrations in serially diluted samples of pooled human plasma.

FIGS. 8A-C. Conventional Simoa assays performed with different SβG concentrations and incubation times for (A) human IL-10 (150 pM SβG for five minutes), (B) human IL-1(3 (150 pM SβG for five minutes), and (C) human Brachyury (300 pM SβG for 15 minutes). Grey dashed lines indicate the calculated LOD for each assay. LOD values for each assay were 575 aM, 2.77 fM, and 1.48 fM for IL-10, IL-1β, and Brachyury, respectively.

FIGS. 9A-C. Overview of CARD-dELISA. A. Target protein molecules are captured on antibody-coated beads, and then protein molecules are labeled with a biotinylated detection antibody and streptavidin-poly-HRP. In the on-bead enzymatic signal amplification step, beads are incubated with tyramide-Alexa Fluor 488. In the presence of hydrogen peroxide, HRP catalyzes radical formation of the tyramide molecule, which then forms a covalently bond with phenol residues on nearby proteins. Only beads containing a full immunocomplex are labeled with the tyramide-Alexa Fluor 488 reagent. B. To encapsulate beads in fibrin hydrogels for imaging, beads are arrayed on a glass slide inside a silicon isolation well, and then a solution of fibrinogen and thrombin is added to the bead array. Beads become immobilized in the fibrin hydrogel as it forms in situ. C. Immobilized bead arrays are imaged using a fluorescence microscope to perform single molecule counting. Beads are identified in brightfield images and bead intensity from fluorescent images is used to determine “on” and “off” beads.

FIGS. 10A-B. Bead immobilization in fibrin hydrogel. A. Each isolation well (7 mm×7 mm×2 mm) contains one sample with beads immobilized in a fibrin hydrogel. B. Brightfield image (10× magnification) of several hundred beads in the fibrin hydrogel (small black dots). This image represents a small region of the entire fibrin hydrogel in a single isolation well. The entire isolation well can be captured in ˜20-25 images at 10× magnification. Scale bar=100 μm.

FIGS. 11A-E. Image analysis and single molecule counting. A-C. Representative microscope images showing a small region of interest with “on” and “off” beads. Images showing (A.) beads (brightfield image) and (B.) fluorescence intensity of tyramide-Alexa Fluor 488 reagent (488 nm fluorescence image) are (C.) overlaid with grey arrows indicating “on” beads. Scale bars=10 μm. Representative histograms of bead fluorescence intensity for (D.) 0 fM and (E.) 50 fM IL-6. The cutoff between “off” and “on” beads is indicated by the grey box in each histogram.

FIG. 12. IL-6 calibration curve using CARD-dELISA. Each data point on the curve represents the average of duplicate measurements. Error bars represent the standard deviation of duplicate measurements. Inset: Comparison of saliva samples measured by CARD-dELISA and conventional Simoa. Data points represent the average of duplicate measurements and error bars represent the standard deviation of duplicate measurements. The dotted line represents exact correlation between the two methods. The Spearman correlation coefficient is 1.00.

FIG. 13. IL-6 calibration curve generated by conventional Simoa. Data points represent the average of duplicate measurements.

### DETAILED DESCRIPTION

Quantitative and ultra-sensitive detection of protein biomarkers in minimally invasive biofluids such as blood or saliva has the potential to revolutionize medical diagnostics with earlier disease diagnoses, treatment monitoring, and disease reoccurrence monitoring. Techniques such as digital enzyme-linked immunosorbent assays (ELISA) and single molecule arrays (Simoa) allow for ultra-sensitive detection of low-abundance biomolecules, including proteins (Rissin et al., Nat. Biotechnol. 2010, 28 (6), 595-599; Leirs et al., Anal. Chem. 2016, 88 (17), 8450-8458; Cohen et al., Chem. Rev. 2019, 119 (1), 293-321), nucleic acids (Song et al., Anal. Chem. 2013, 85 (3), 1932-1939; Cohen et al., Nucleic Acids Res. 2017, 45 (14), e137-e137), and other biologically-relevant small molecules (Wang et al., J. Am. Chem. Soc. 2018, 140 (51), 18132-18139; Wang and Walt, Chem. Sci. 2020. doi.org/10.1039/D0SCO2552F), by isolating and counting individual molecules in microwell arrays (Rondelez et al., Nat. Biotechnol. 2005, 23 (3), 361-365; Rissin et al., Nano Lett. 2006, 6 (3), 520-523; Cohen and Walt, Annu. Rev. Anal. Chem. 2017, 10 (1), 345-363) or microfluidic droplets (Kim et al., Lab. Chip 2012, 12 (23), 4986; Witters et al., Lab. Chip 2013, 13 (11), 2047; Yelleswarapu et al., Proc. Natl. Acad. Sci. 2019, 116 (10), 4489-4495; Cohen et al., ACS Nano 2020, 14, 8, 9491-9501).

Ultra-sensitive protein detection can be achieved in Simoa as follows: first, individual protein molecules are captured on antibody-coated paramagnetic beads. An excess number of beads compared to the number of protein molecules is used to ensure each bead binds either zero or one protein molecule. Then, bound protein molecules are labeled with a biotinylated detection antibody and streptavidin-conjugated enzyme. Finally, beads are resuspended in a solution containing a fluorogenic enzyme substrate and loaded into microwell arrays. The arrays are sealed with oil and a localized concentration of fluorescent product is produced only in wells with a full immunocomplex. Single molecule counting is performed by counting active wells and the fraction of “on” beads to the total number of beads is calculated, and then converted to average enzyme per bead (AEB) to produce calibration curves. Recently, Simoa has been implemented in numerous clinical applications (Wu et al., Crit. Rev. Clin. Lab. Sci. 2020, 57 (4), 270-290), including neurological and neurodegenerative diseases (Mattsson et al., JAMA Neurol. 2017, 74 (5), 557; Shahim et al., JAMA Neurol. 2014, 71 (6), 684; Gill et al., Neurology 2018, 91 (15), e1385-e1389; Ng et al., Clin. Transl. Neurol. 2019, 6 (3), 615-619), oncology (Wilson et al., Clin. Chem. 2011, 57 (12), 1712-1721; Shi et al., Nature 2019, 569 (7754), 131-135; Olsen et al., J. Immunol. Methods 2018, 459, 63-69), and infectious diseases (Leirs et al., 2016, supra; J. Clin. Microbiol. 2018, 56 (8); Anderson et al., Clin. Infect. Dis. 2018, 67 (1), 137-140; Ahmad et al., Sci. Transl. Med. 2019, 11 (515), eaaw8287). Simoa can be used to detect proteins in the femtomolar (fg/mL) or subfemtomolar range of concentrations.

We have developed innovative, simple single molecule measurement platforms that can detect low- to mid-attomolar protein concentrations. By addressing the challenge of low efficiencies in sampling rare target molecules in digital ELISA approaches, we enhanced sensitivity by up to 25-fold over the current Simoa technology, which is presently the gold standard for ultrasensitive protein detection. The attomolar limits of detection (LODs) achieved by the present methods correspond to an over 10,000-fold increase in sensitivity over conventional immunoassays. Localization of a non-diffusible amplified signal to each bead eliminates the need for signal compartmentalization into microwells or droplets.

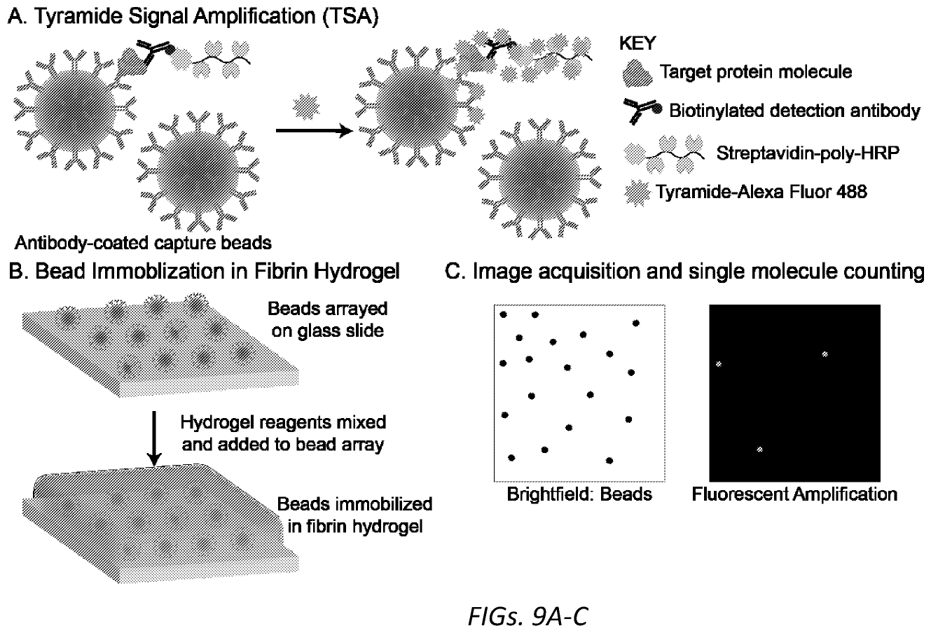

In some embodiments, the methods include direct dropcasting of all the beads onto a surface, e.g., a slide, for rapid drying and formation of a monolayer film, or immobilization of the beads in a layer of hydrogel. One exemplary method uses on-bead signal generation combined with bead dropcasting into a monolayer film for single molecule counting. One embodiment of these methods is referred to as dSimoa herein. In addition, provided herein are methods that use Tyramide Signal Amplification (TSA), a method for Catalyzed Reporter Deposition (CARD), for on-bead signal generation (FIG. 9A), followed by bead immobilization in fibrin hydrogels (FIG. 9B) and imaging for single molecule counting (FIG. 9C). Some embodiments of this method are referred to herein as CARD digital ELISA (CARD-dELISA). By localizing a non-diffusible fluorescent signal to each bead carrying a target molecule, this platform not only eliminates the need for bead loading into microwells or droplets for signal compartmentalization, but also enables significantly more beads to be analyzed for improved sampling efficiency and thereby enhances sensitivity.

This simple approach allows 40-50% on average of the total assay beads to be analyzed—an eight- to ten-fold increase over the ˜5% sampling efficiency of the current Simoa technology. At low sample concentrations, particularly with capture efficiencies well below 100% (˜1-3% across all capture and labeling steps in the present assays developed in this work), improved sampling is critical for minimizing Poisson noise-associated measurement CVs. Although some beads may be lost during washing steps or excluded from analysis if overlapping or aggregated, the experimental results showed that analyzing 20% of the total assay beads improved LODs by about an order of magnitude, with small further improvements in measurement CVs and the LOD as more beads were analyzed. The significantly improved sampling efficiency of the present methods also allows the use of fewer assay beads compared to conventional Simoa, increasing the fraction of “on” beads” and thereby the signal to background. Further improvements in sensitivity can be attained by using affinity reagents with lower dissociation constants and decreasing nonspecific binding of the affinity reagents and streptavidin-DNA label. With the development of better affinity reagents and methods to reduce nonspecific binding, the present methods can potentially detect down to zeptomolar protein concentrations.

With attomolar sensitivity, the present methods can pave the way towards discovery of new biomarkers and biological mechanisms underlying various diseases. As a proof of principle, we demonstrated that the present methods can measure low concentrations of the T-box-family transcription factor Brachyury that were previously undetectable by the current Simoa technology in plasma samples from chordoma patients. While Brachyury has been shown to be highly overexpressed in the tumors of chordoma patients, its levels in plasma have not been assessed.²⁶⁻³⁰ As the diagnosis of chordoma requires an invasive needle or incisional biopsy into the skull base or spine, a blood-based test would provide a significantly lower-risk diagnostic procedure and potentially facilitate early diagnosis of chordoma.^{31,32} While our measurements were performed in only a small sample cohort, the significantly improved detectability of Brachyury in chordoma patient plasma samples using the present methods opens new possibilities for a potential blood test and the discovery of new biological mechanisms. Achieving an order of magnitude or more improvement in sensitivity with the present methods also holds important implications for the discovery of new blood-based biomarkers for many other cancer types and neurological disorders. A diagnostic blood test for neurodegenerative diseases such as Alzheimer's and Parkinson's diseases would prove especially critical for widespread screening and early diagnosis, which are currently very difficult due to the need for highly invasive lumbar punctures. In many cases where biomarker levels become detectable only after significant disease progression, the enhanced sensitivity of the present methods can accelerate disease diagnosis in early stages for improved health outcomes.

Importantly, the present methods also increase the simplicity of digital bioassay signal readout, which upon further development can potentially be integrated into a POC platform and thus address challenges of low sensitivity in current POC diagnostics. While increasing sampling efficiency for enhanced sensitivity in digital immunoassays has also been demonstrated in bead droplet arrays and droplet digital ELISA methods, these methods introduce additional complexity in fabrication and processing steps.^{13,14} In contrast, the digital readout process for the present methods requires only a microscope slide for bead loading and a simple optical setup, with no additional materials or complex instrumentation needed. Furthermore, the dropcasting process is remarkably simple and rapid. The dropcast method simplifies the single molecule detection readout process and increases cost-effectiveness compared to current microwell- or droplet-based digital ELISA methods. The enhanced sensitivity of the present methods enables the detection of various biomarkers that exist at very low concentrations and have not previously been measured in easily accessible biofluids such as saliva and urine. An additional interesting aspect of the present methods is the long-term signal stability in the dropcast films, which increases flexibility in the assay process. For instance, in resource-limited settings where a suitable optical setup may not be readily available, the dropcast films can be easily shipped to facilities for imaging and analysis, with no signal loss for at least one month.

The present methods can be adapted for POC applications, including integration into a microfluidic device for sample processing and incorporation of portable imaging. For example, the sample processing workflow can be automated in a microfluidic system, which combined with the single molecule resolution and high sampling efficiency of the present methods, can potentially reduce assay times while still achieving high sensitivities for detecting low abundance biomarkers that are currently undetectable by existing POC platforms. Furthermore, due to the rapid kinetics of RCA and the reduced diffusional distances in small microfluidic reaction volumes, the times for each sample processing step, including RCA, can be shortened. Although RCA was carried out for one hour in this example set forth below, detectable signals were observable after fifteen minutes. RCA signal amplification time can be further reduced by increasing the spatial density of fluorescent labels on each concatemer. As several automated and streamlined microfluidic-based methods have been developed for bead-based immunoassays, the present sample processing steps can be incorporated into an automated microfluidic platform.^{5, 33-36} In addition, many portable fluorescence imaging platforms have been developed, including smartphone attachments for imaging single fluorescent nanoparticles and RCA products, that can be readily adapted for the present methods.^{5, 37-41} In some embodiments, multiple frames can be used to completely capture each dropcast film, and shorter imaging times can also be used, e.g., using an automated handheld reader or a wide field-of-view camera. Moreover, as supported by the sampling analysis of the results using the present methods, only ˜20% of the total assay beads may need to be imaged to achieve close to maximal sensitivity. Integration of the present methods into an ultrasensitive, portable, and automated platform be used to facilitate widespread screening, early detection, and monitoring of many diseases, including infectious diseases such as the recent SARS-CoV-2 pandemic and tuberculosis, traumatic brain injuries, and myocardial infarction.

The present versions of digital ELISA have a simplified workflow and allow for ultra-sensitive quantitation of proteins in biological fluids. In some embodiments, for example, CARD-dELISA uses tyramide signal amplification for on-bead enzymatic signal generation.

The present methods can include bead encapsulation in fibrin hydrogels and imaging for single molecule counting. CARD-dELISA shows good sensitivity and dynamic range, indicating that CARD-dELISA is a simpler yet robust alternative to conventional Simoa. Furthermore, CARD-dELISA eliminates the need for some of the expensive instrumentation and consumables required in conventional Simoa, demonstrating that CARD-dELISA is suited for incorporation into a point-of-care digital ELISA platform. Future work will include developing a combined point-of-care CARD-dELISA platform with integrated sample processing (i.e. target capture, labeling, and on-bead signal amplification)^{13,32,33}, bead imaging, and data analysis. For bead imaging, we will incorporate a compact microscope module into the platform with sensitivity for measuring micro- and nanoscale objects. The microscope module will include an LED light source, appropriate filters and lenses, and imaging will be performed using a smart phone camera³⁴⁻³⁶ or compact CMOS.³⁷ Additionally, we will continue to optimize and improve CARD-dELISA to increase the assay sensitivity and decrease the total assay time. Although, CARD-dELISA is currently ˜10X less sensitive than conventional Simoa, automation of CARD-dELISA into a fully integrated point-of-care device will likely lower the CVs (coefficient of variation) of background signal and therefore improve assay sensitivity. Once integrated into a point-of-care device, CARD-dELISA is a promising platform for triage tests or early diagnostics for diseases such as TB, sepsis, or mild traumatic brain injury.

Assay Methods

In the present methods, a sample in solution is contacted with a plurality of beads conjugated to capture moieties that bind to a biomolecule of interest, under conditions that allow binding of the sample to the beads to form a bead-biomolecule complex. Once the complex is formed, the methods include contacting the biomolecule with a second capture moiety that allows for generation of an on-bead, non-diffusible detectable signal, e.g., a fluorescent signal, that allows detection of each bead carrying a target molecule, and then generating the amplified signal. The methods can include removal of unbound beads.

The methods then include immobilizing the beads, e.g., by dropcasting the solution comprising the beads onto a surface to form a film (e.g., formation of a thin film by dropping the solution onto a flat surface followed by evaporation of the solution), or by catalyzing gelation of the solution. The surfaces can include, e.g., slides, chips, or flowcells adapted for detection, e.g., by imaging. Finally, the methods include detecting the signal from the beads, and optionally determining a number and/or percentage of beads that comprise bead-biomolecule complexes.

In general, the sample is maintained intact before imaging, e.g., is not subdivided or compartmentalized into individual wells before imaging. The present methods eliminate the need for signal compartmentalization into microwells or droplets.

Sample

As used herein the term “sample”, when referring to the material to be tested for the presence of a biomolecule of interest marker using the method of the invention, includes inter alia tissue, whole blood, plasma, serum, urine, sweat, saliva, breath, exosome or exosome-like microvesicles (U.S. Pat. No. 8,901,284), lymph, feces, cerebrospinal fluid, ascites, bronchoalveolar lavage fluid, pleural effusion, seminal fluid, sputum, nipple aspirate, post-operative seroma or wound drainage fluid. The type of sample used may vary depending upon the identity of the biological marker to be tested and the clinical situation in which the method is used. Various methods are well known within the art for the identification and/or isolation and/or purification of a biological marker from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

Beads

The present methods include the use of micro or nanoparticle beads conjugated to capture moieties that bind to a desired biomolecule. The micro- and/or nanoparticles (e.g., microbeads) can be made of various materials. In general, any polymeric or plastic materials can be used to create the microparticles, microbeads, or nanoparticles, including materials such as polystyrene and polyethylene, for example. In some embodiments, microparticles can be formed of biologically-compatible polymer materials such as polyacrylates, polymethacrylates, and/or polyamides.

In certain embodiments, metallic, metal-oxide, semiconductor, and/or semiconductoroxide micro- and/or nanoparticles formed from one or more of Au, Ag, Pt, Al, Cu, Ni, Fe, Cd, Se, Ge, Pd, Sn, iron oxide, TiO₂, Al₂O₃, and SiO₂ can be made in many sizes and used. For example, monocrystalline iron oxide nanoparticles (MIONs) and crosslinked iron oxide (CLIO) particles can be used. In some embodiments, the beads are paramagnetic. Suitable beads include, but are not limited to, magnetic beads (e.g., paramagnetic beads), plastic beads, ceramic beads, glass beads, silica beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, and TEFLON® beads. In some embodiments, spherical beads are used, but non-spherical or irregularly-shaped beads may be used.

In some embodiments, the beads are as described in “Ultra-sensitive detection of molecules on single molecule arrays,” D. Duffy, E. Ferrell, J. Randall, D. Rissin, D. Walt. U.S. Pat. No. 8,222,047, Jul. 17, 2012; “Methods and arrays for target analyte detection and determination of target analyte concentration in solution,” D. M. Rissin, D. R. Walt. U.S. Pat. No. 8,460,879, Jun. 11, 2013; “Methods and arrays for target analyte detection and determination of reaction components that affect a reaction” David Walt, David Rissin, Hans-Heiner Gorris. U.S. Pat. No. 8,492,098, Jul. 23, 2013; “Ultra-sensitive detection of molecules on single molecule arrays,” David C. Duffy, Evan Ferrell, Jeffrey D. Randall, David M. Rissin, David R. Walt. U.S. Pat. No. 8,846,415, Sep. 30, 2014; “Ultra-sensitive detection of molecules or particles using beads or other capture objects,” D. C. Duffy, D. M. Rissin, D. R. Walt, D. Fournier, C. Kan. Quanterix Corporation. U.S. Pat. No. 9,310,360, Apr. 12, 2016; “Methods and arrays for target analyte detection and determination of target analyte concentration in solution,” D. R. Walt, D. M. Rissin. U.S. Pat. No. 9,395,359, Jul. 19, 2016; or “Ultra-sensitive detection of molecules or particles using beads or other capture objects”, D. C. Duffy, D. M. Rissin, D. R. Walt, D. Fournier, C. Kan. Quanterix Corporation. U.S. Pat. No. 9,482,662, Nov. 1, 2016; or WO2020037130.

Capture and Binding Moieties

The beads are coated with, e.g., conjugated to, capture moieties that bind to a biomolecule of interest. In addition, binding moieties are used to detect beads bound to biomolecules and amplify the signal therefrom.

In some embodiments, the capture or binding moiety is an antibody or antigen-binding portion thereof or an aptamer that binds to the biomolecule, e.g., wherein the biomolecule is a protein or peptide. In some embodiments, the capture or binding moiety is an oligonucleotide that is complementary to a portion of a nucleic acid of interest. In some embodiments, the capture or binding moiety is a ligand-binding portion of a protein, e.g., of a receptor, wherein the biomolecule is a molecule such as a hormone.

The capture or binding moiety is capable of specifically binding to or otherwise specifically associating with a capture moiety or a target analyte. A capture or binding moiety can be conjugated, captured, attached, bound, or affixed to a capture moiety. For example, in some embodiments, a capture or binding moiety is an antibody (e.g., a full-length antibody {e.g., an IgG, IgA, IgD, IgE, or IgM antibody) or an antigen-binding antibody fragment {e.g., an scFv, an Fv, a dAb, a Fab, an Fab′, an Fab′2, an F(ab′)2, an Fd, an Fv, or an Feb)), an aptamer, an antibody mimetic {e.g., an affibody, an affilin, an affimer, an affitin, an alphabody, an anticalin, an avimer, a DARPin, a fynomer, a Kunitz domain peptide, a monobody, or a nanoCLAMP), an antibody IgG binding protein {e.g., protein A, protein G, protein L, or recombinant protein A/G), a polypeptide, a nucleic acid, or a small molecule. For example, in some embodiments, a capture or binding moiety binds to an Fc region of an antibody.

In some embodiments, the methods include the use of a capture moiety that binds to the biomolecule; and a binding moiety that binds to the capture moiety. In some embodiments, the methods include the use of a capture moiety that binds to the biomolecule; a first binding moiety that binds to the capture moiety; and a second binding moiety or signal amplification moiety that binds to the first binding moiety. One or more of the capture moiety, the binding moiety, or the second binding moiety/signal amplification moiety, can include or generate a detectable label. For example, in some embodiments, the binding moiety comprises biotin, and the second binding moiety/signal amplification moiety is a streptavidin labeled horseradish peroxidase (HRP) enzyme that binds before signal generation. The sample can be contacted with the capture and binding moieties simultaneously (e.g., in the same solution), or can be contacted sequentially, e.g., with the capture moieties and then the binding moieties. The methods can include removing any unbound reagents, e.g., beads, capture and/or binding moieties, before detection.

In some embodiments, the capture or binding moieties are as described in “Ultra-sensitive detection of molecules on single molecule arrays,” D. Duffy, E. Ferrell, J. Randall, D. Rissin, D. Walt. U.S. Pat. No. 8,222,047, Jul. 17, 2012; “Methods and arrays for target analyte detection and determination of target analyte concentration in solution,” D. M. Rissin, D. R. Walt. U.S. Pat. No. 8,460,879, June 11th, 2013; “Methods and arrays for target analyte detection and determination of reaction components that affect a reaction” David Walt, David Rissin, Hans-Heiner Gorris. U.S. Pat. No. 8,492,098, Jul. 23, 2013; “Ultra-sensitive detection of molecules on single molecule arrays,” David C. Duffy, Evan Ferrell, Jeffrey D. Randall, David M. Rissin, David R. Walt. U.S. Pat. No. 8,846,415, Sep. 30, 2014; “Ultra-sensitive detection of molecules or particles using beads or other capture objects,” D. C. Duffy, D. M. Rissin, D. R. Walt, D. Fournier, C. Kan. Quanterix Corporation. U.S. Pat. No. 9,310,360, Apr. 12, 2016; “Methods and arrays for target analyte detection and determination of target analyte concentration in solution,” D. R. Walt, D. M. Rissin. U.S. Pat. No. 9,395,359, Jul. 19, 2016; or “Ultra-sensitive detection of molecules or particles using beads or other capture objects”, D. C. Duffy, D. M. Rissin, D. R. Walt, D. Fournier, C. Kan. Quanterix Corporation. U.S. Pat. No. 9,482,662, Nov. 1, 2016; or WO2020037130.

Biomolecules

In some embodiments, the biomolecule of interest is a protein, peptide, nucleic acid, virus, cell surface molecule, metabolite, or small molecule.

By “biomolecule” is meant any atom, molecule, ion, molecular ion, compound, particle, cell, virus, complex, or fragment thereof to be either detected, measured, quantified, or evaluated. A target analyte may be contained in a sample {e.g., a liquid sample {e.g., a biological sample or an environmental sample)). Exemplary target analytes include, without limitation, a small molecule (e.g., an organic compound, a steroid, a hormone, a hapten, a biogenic amine, an antibiotic, a mycotoxin, an organic pollutant, a nucleotide, an amino acid, a monosaccharide, or a secondary metabolite), a protein (including a glycoprotein or a prion), a nucleic acid {e.g., a modified nucleic acid or an miRNA), a polysaccharide, a lipid, an extracellular vesicle, a glycan, a toxin, a fatty acid, a cell, a gas, a therapeutic agent, an organism (e.g., a pathogen), or a virus. The target analyte may be naturally occurring or synthetic. In some embodiments, a target analyte is an interferon, e.g., interferon g (IFNg). In some embodiments, a target analyte is an interleukin, e.g., interleukin 2 (IL-2).

The terms “nucleic acid” and “polynucleotide,” as used interchangeably herein, refer to at least two covalently linked nucleotide monomers. The term encompasses, e.g., deoxyribonucleic acid (DNA), ribonucleic acid (RNA), hybrids thereof, and mixtures thereof. Nucleotides are typically linked in a nucleic acid by phosphodiester bonds, although the term“nucleic acid” also encompasses nucleic acid analogs having other types of linkages or backbones {e.g., phosphorothioate, phosphoramide, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, and the like). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases.

By “protein” herein is meant at least two covalently linked amino acids, which includes proteins, polypeptides, oligopeptides and peptides. The protein may be made up of naturally occurring amino acids and peptide bonds, or synthetic peptidomimetic structures. Thus, “amino acid,” or “peptide residue,” as used herein, means both naturally occurring and synthetic amino acids. For example, homo-phenylalanine, citrulline and norleucine are considered amino acids for the purposes of the invention. The side chains may be in either the (R) or the (S) configuration. In some embodiments, the amino acids are in the (S) or L-configuration. If non-naturally occurring side chains are used, non-amino acid substituents may be used, for example to prevent or retard in vivo degradation. The term “portion” includes any region of a protein, such as a fragment {e.g., a cleavage product or a recombinantly-produced fragment) or an element or domain (e.g., a region of a polypeptide having an activity) that contains fewer amino acids than the full-length or reference polypeptide {e.g., about 5%, 10%, 15%, 20%, 30%, 40%, 50%, 60%, 70%, 80%, 90%, 95%, or 99% fewer amino acids).

The term “small molecule,” as used herein, means any molecule having a molecular weight of less than 5000 Da. For example, in some embodiments, a small molecule is an organic compound, a steroid, a hormone, a hapten, a biogenic amine, an antibiotic, a mycotoxin, a cyanotoxin, a nitro compound, a drug residue, a pesticide residue, an organic pollutant, a nucleotide, an amino acid, a monosaccharide, or a secondary metabolite. See also WO2020037130.

Detection Methods

The present methods include on-bead signal amplification for single-molecule signal generation. The signal can be any detectable signal, e.g., optically detectable labels such as fluorescent or chemiluminescent, or colorimetric, or can be an other label, e.g., gold beads or other label detectable by non-optical assays (e.g., using surface plasmon resonance or other methods). In some embodiments, the methods use rolling circle amplification of a concatemer; the generated DNA concatemer attached to each immunocomplex can be hybridized with a large number of complementary fluorescently labeled DNA probes for visualization. In these methods, the sensitivity can be tuned by increasing (more sensitive) or decreasing (less sensitive) RCA time.

Other nucleic acid amplification methods can also be used, e.g., hybridization chain reaction, Enzyme-catalyzed proximity labeling (PL) polymerization (see, e.g., Branon et al., Nat Biotechnol. 2018 October; 36(9):880-887); polymerization-based signal amplification (e.g., visible-light-induced polymerization, e.g., as described in Badu-Tawiah et al., Lab Chip, 2015, 15, 655); magnetic bead-quantum dot immunoassays (Kim et al., ACS Sens. 2017, 2, 6, 766-772); or immunosignal hybridization chain reaction (isHCR) (Lin et al., Nat Methods. 2018 April; 15(4):275-278). Branched DNA can also be used.

Alternatively, the methods can include using Tyramide Signal Amplification (TSA). TSA, also referred to as Catalyzed Reporter Deposition (CARD), is a highly sensitive method enabling the detection of biomolecules present in low abundance. TSA is used in immunohistochemistry and in situ hybridization experiments, and has been used for digital ELISA (Akama et al., Anal. Chem. 2016, 88 (14), 7123-7129). In TSA, HRP (e.g., bound to a second binding moiety) catalyzes the conversion of labeled tyramide into a reactive radical which then covalently binds to nearby tyrosine residues, generating a high-density detectable signal.

Other amplification chemistries can also be used, e.g., as described in Dunbar and Das, J Clin Virol. 2019 June; 115: 18-31, e.g., branched DNA assays (bDNA).

In some embodiments, the methods include contacting the binding moiety with a pre-amplified signal such as a labeled polymer or nanoparticle; see, e.g. Tang et al., Analyst, 2013,138, 981-990; Hansen et al., Anal Bioanal Chem. 2008 September; 392(1-2): 167-175; Wu et al., Chem 2, 760-790, Jun. 8, 2017; Skaland et al., Applied immunohistochemistry & molecular morphology: AIMM/official publication of the Society for Applied Immunohistochemistry 18(1):90-6 (2009); Gormley et al., Nano Lett. 2014, 14, 11, 6368-6373 (radicals generated by either enzymes or metal ions are polymerized to form polymers that entangle multiple gold nanoparticles (AuNPs)); Dye-Loaded Polymeric Nanoparticles (e.g., as described in Melnychuk and Klymchenko, J. Am. Chem. Soc. 2018, 140, 34, 10856-10865).

Imaging or other methods suitable for the selection detactable label can be used. In some embodiments, the beads are immobilized in a monolayer and a single z-section imaging can be used; in embodiments where the beads are not in a monolayer, the methods can include imaging different z sections.

Hydrogel

In some embodiments, the methods include gelation of a layer of hydrogel to immobilize individual beads before signal detection. Methods for catalyzing gelation of a hydrogel are known in the art. Hydrogels can comprise, e.g., fibrin, fibrinogen, cellulose, collagen, gelatin, agarose, and hyaluronic acid, or synthetic hydrogels such as polyhydroxyethylmethacrylate (poly(HEMA)), polyethylene glycol (PEG), or acrylamide. See, e.g., Ahmed, J Adv Res. 2015 March; 6(2):105-21.

Kits

Also provided herein are kits for use in a method described herein, e.g., comprising beads and reagents as described herein.

### Examples

The invention is further described in the following examples, which do not limit the scope of the invention described in the claims.

### Example 1. Ultrasensitive Detection of Attomolar Protein Concentrations by Dropcast Single Molecule Assays

We have developed a simple, ultrasensitive single molecule detection platform that enhances sensitivity by up to 25-fold over the current state-of-the-art digital ELISA technology. By improving sampling of rare target molecules, this approach enables protein detection in the attomolar range, thus opening a window into a wide range of potential disease biomarkers that were previously unmeasurable. Importantly, the present methods also simplify the digital assay readout process and is therefore more amenable to future integration into a POC system. The platform can also be readily adopted to measure other disease-related biomolecules, including microRNAs and small molecules, in simpler and more sensitive assays compared to the previously developed Simoa assays.^{42, 43} By measuring very low concentrations of biomolecules that are undetectable by current methods, the present methods provide a platform for ultrasensitive detection that can facilitate early disease diagnosis.

Methods

Materials. All antibodies, recombinant proteins, and DNA sequences used in this study are listed in the Supplementary Information. DNA primer, template, and probe were obtained from Integrated DNA Technologies or MilliporeSigma. Conjugation and assay buffers, as well as dye-encoded carboxylated 2.7-μm paramagnetic beads (Homebrew Multiplex Beads 488), were purchased from Quanterix Corporation.

Preparation of antibody-coated capture beads. For each target, capture antibody was buffer exchanged into Bead Conjugation Buffer (Quanterix), using a 50K Amicon centrifugal filter (0.5 mL, MilliporeSigma). Bead Conjugation Buffer was added to antibody solution in the filter up to 500 μL, followed by centrifugation at 14,000×g for five minutes. The effluent was discarded and the process was repeated twice. Buffer-exchanged antibody was recovered by inverting the filter into a new tube and centrifuging at 1000×g for two minutes, followed by a 50 μL Bead Conjugation Buffer rinse and a second centrifugation at 1000×g for two minutes. Antibody concentration was measured with a NanoDrop spectrophotometer, and antibody was diluted to 0.5 mg/mL (IL-10), 0.3 mg/mL (Brachyury), or 0.2 mg/mL (IL-1β) in Bead Conjugation Buffer for subsequent bead coupling. 2.8×10⁸dye-encoded paramagnetic beads were washed three times with 200 μL Bead Wash Buffer (Bead Conjugation Buffer) and two times with 200 μL Bead Conjugation Buffer, and resuspended in 190 μL cold Bead Conjugation Buffer. A 1 mg vial of 1-ethyl-3-(3-dimethylaminopropyl) carbodiimide hydrochloride (EDC) (Thermo Fisher Scientific) was then reconstituted in 100 μL cold Bead Conjugation Buffer, and 10 μL was immediately added to the beads. The beads were activated for 30 minutes under shaking. After activation, the beads were washed with 200 μL cold Bead Conjugation Buffer, resuspended in 200 μL of capture antibody solution, and placed on a shaker for two hours for antibody coupling. The antibody-coupled beads were subsequently washed two times with 200 μL Bead Wash Buffer and blocked with 200 μL Bead Blocking Buffer (Quanterix) for 30 minutes under shaking. After blocking, the beads were washed with 200 μL Bead Wash Buffer and then with 200 μL Bead Diluent (Quanterix), before resuspension in 200 μL Bead Diluent. For IL-1(3, the EDC activation and antibody coupling steps were performed at 4° C., with 4.2×10⁸ beads, 9 μL EDC for bead activation, and 300 μL 0.2 mg/mL antibody for conjugation. A Beckman Coulter Z1 Particle Counter was used to count the beads, which were stored at 4° C. for subsequent use in assays.

Preparation of streptavidin-DNA conjugate. The RCA template (MilliporeSigma) was first annealed to a 5′ azide-modified primer (Integrated DNA technologies) by heating a solution of 45 μL 100 μM primer, 54 μL 100 μM template, and 26.6 μL 5×NEBNext® Quick Ligation reaction buffer (New England Biolabs) at 95° C. for two minutes and allowing to slowly cool to room temperature over 90 minutes. The template was then ligated by adding 7.5 μL T4 DNA ligase (2,000,000 units/mL, New England Biolabs) and incubating the reaction at room temperature for three hours. The ligation reaction was then buffer exchanged into PBS with 1 mM EDTA using a Zeba™ spin desalting column (7K MWCO, Thermo Fisher Scientific). Streptavidin (Biolegend 280302) was buffer exchanged into phosphate-buffered saline (PBS) with a 10K Amicon centrifugal filter (0.5 mL, MilliporeSigma), following the same buffer exchange procedure as described above for capture antibodies, and then diluted to 1 mg/mL in PBS. Dibenzocyclooctyne-PEG4-N-hydroxysuccinimidyl ester (DBCO-PEG4-NHS, 1 mg, MilliporeSigma) was dissolved in 200 μL dimethyl sulfoxide, and a 20-fold molar excess was added to the buffer-exchanged streptavidin. The conjugation reaction was allowed to incubate for 30 minutes at room temperature and then purified with a 10K Amicon centrifugal filter. The conjugated streptavidin was washed with PBS with 1 mM EDTA in five centrifugations at 14,000xg for five minutes followed by one centrifugation at 14,000×g for 15 minutes. The purified DBCO-conjugated streptavidin was then recovered by inverting the filter and centrifuging at 1000xg for two minutes. Annealed primer-template was added to the DBCO-conjugated streptavidin at a two-fold molar excess and the conjugation reaction was allowed to proceed overnight at 4° C. The streptavidin-DNA conjugate was then stored in aliquots at −80° C. with 0.1% bovine serum albumin (BSA), 5 mM EDTA, and 0.02% sodium azide, without further purification.

Dropcast single molecule assays. All dSimoa assays were performed in 96-well plates (Greiner Bio-One, 655096). Antibody-coated beads, recombinant proteins, and biotinylated detector antibodies were diluted in Sample Diluent (Quanterix) to the desired concentrations. Detector antibody and streptavidin-DNA concentrations for each assay are listed in the Supplementary Information. For each assay, 10 antibody-coated beads (100,000 beads total) and 10 μL biotinylated detector antibody were added to 100 μL of protein sample. The plate was then sealed and shaken for one hour for immunocomplex formation. The beads were washed six times with System Wash Buffer 1 (Quanterix) using a BioTek 405 TS Microplate Washer, followed by resuspension in 100 μL streptavidin-DNA conjugate diluted in Sample Diluent with 5 mM ethylenediaminetetraacetic acid (EDTA). The plate was shaken for 15 minutes for streptavidin-DNA labeling of the immunocomplexes and then washed eight times with System Wash Buffer 1 using the microplate washer. After washing, the beads were transferred to a new 96-well plate and washed an additional time with 200 μL System Wash Buffer 1 before resuspending in 60 μL RCA solution. The RCA solution consisted of 0.5 mM deoxynucleotide mix (New England Biolabs), 0.33 U/uL phi29 DNA polymerase (Lucigen), 0.2 mg/mL BSA, 1 nM ATTO 647N-labeled DNA probe (Integrated DNA Technologies), and 0.1% Tween-20 in a reaction buffer comprising 50 mM Tris-HCl (pH 7.5), 10 mM (NH₄)₂SO₄, and 10 mM MgCl₂. Dithiothreitol (DTT) was removed from the phi29 polymerase solution received from the manufacturer using a Zeba™ spin desalting column (7K MWCO, Thermo Fisher Scientific). RCA was performed at 37° C. for one hour with shaking of the plate, after which 150 μL PBS with 5 mM EDTA was added to each sample to stop the RCA reaction. The beads were then washed two times with 200 μL dropcast buffer (50 mM Tris-HCl, 50 mM NaCl, 0.1% Tween-20, 0.5% BSA) and concentrated to 10-15 μL before resuspending and dropcasting onto a microscope slide via manual pipetting. The dropcast beads were allowed to dry for ten to fifteen minutes to form monolayer films.

For saliva samples, pooled human saliva (BioIVT) was centrifuged at 13,150×g for 20 minutes at 4° C. In dilution linearity experiments, the desired volume of supernatant was serially diluted 2-to 32-fold in Sample Diluent with protease inhibitor (Halt™ Protease Inhibitor Cocktail, Thermo Fisher Scientific). For spike and recovery experiments, recombinant human IL-10 protein was spiked into 4-fold diluted saliva samples at 100, 10, and 1 fM.

Plasma samples from chordoma patients were obtained from Dr. Sandro Santagata and Dr. Keith Ligon (Brigham and Women's Hospital) and centrifuged at 2000×g for 10 minutes at 4° C., and the supernatant was aliquoted to prevent freeze-thaw cycles. Commercial plasma and serum samples were obtained from BioIVT. All samples were diluted eight-fold in Sample Diluent for measurements.

Imaging and analysis. Brightfield and fluorescent images of the dropcast bead films were acquired using an Olympus IX81 inverted microscope, with a scientific CMOS camera (ORCA-Flash4.0 LT+, Hamamatsu) and 10× objective. Fluorescence images obtained with a GFP filter (1 s exposure) were used to locate the dye-encoded beads, while fluorescence images obtained with a Cy5 filter (1 s exposure) were used to identify “on” versus “off” beads. Commercial software (cellSens) was used to control the stage and camera. Brightfield and fluorescence images were acquired for each frame, and multiple frames were acquired to capture the entire dropcast film, excluding the film edges. About 20-25 frames were acquired per dropcast film, with average total imaging times of approximately 15 minutes.

Image analysis was performed in MATLAB. The beads were first located in the 488 nm fluorescent image using a disk-shaped morphological structuring element, with top-hat filtering to correct for uneven illumination. Overlapping or aggregated beads were separated by watershed segmentation, and any remaining aggregated beads were removed by a size cutoff. The maximum signal intensity on each identified bead was determined in the corresponding Cy5 fluorescent image, which first underwent a top-hat filter to correct for uneven illumination. A Gaussian distribution was fitted to the bead fluorescent intensities, and the cutoff intensity value for an “on” versus “off” bead was determined as five standard deviations above the mean of the distribution. Thus, all beads with intensities above the cutoff value were counted as “on” beads. The fraction of on beads was calculated as the total number of “on” beads over the total number of beads, and the average molecule per bead (AMB) was subsequently calculated from the Poisson distribution.

Calibration curves were fit using a four parameter logistic (4PL) fit in GraphPad Prism and used to determine unknown sample concentrations. The R² values of the calibration curve fits can be found in the Supplementary Information. All measurements were performed in 3-4 replicates, except dilution linearity and spike and recovery assays, which were performed in duplicates. The limit of detection (LOD) of each assay was calculated as the concentration corresponding to three standard deviations above the background AEB.

Simoa assays. Conventional Simoa assays were performed on an HD-X Analyzer (Quanterix), using the same antibody-coated capture beads (500,000 beads per assay) and biotinylated detector antibodies at the same concentrations as in the corresponding dSimoa assays and 100 μL sample volumes. Streptavidin-β-galactosidase (SβG) Concentrate (Quanterix) was diluted in SβG Diluent (Quanterix) to the desired concentration. The same incubation time of one hour was used for the antibody capture step, in which the beads, sample, and detector antibody are incubated for immunocomplex sandwich formation. For each target, two assay conditions were performed: one assay with the same SβG concentrations and incubation times as in the corresponding dSimoa assay, and one assay with a standard SβG concentration and incubation time used on the HD-X (150 pM SβG for five minutes). Beads, detector antibody, and SβG were placed in plastic bottles (Quanterix) and samples were added to a 96-well plate, all of which were loaded into the HD-X Analyzer. The enzyme substrate (resorufin β-D-galactopyranoside), Wash Buffer 1, Wash Buffer 2, and Simoa Sealing Oil were loaded into the HD-X Analyzer according to the manufacturer's instructions. All assay steps, image analyses, and calculations of average enzyme per bead (AEB) were automated, as previously described in detail.¹³

Results

Development of Dropcast Single Molecule Assays

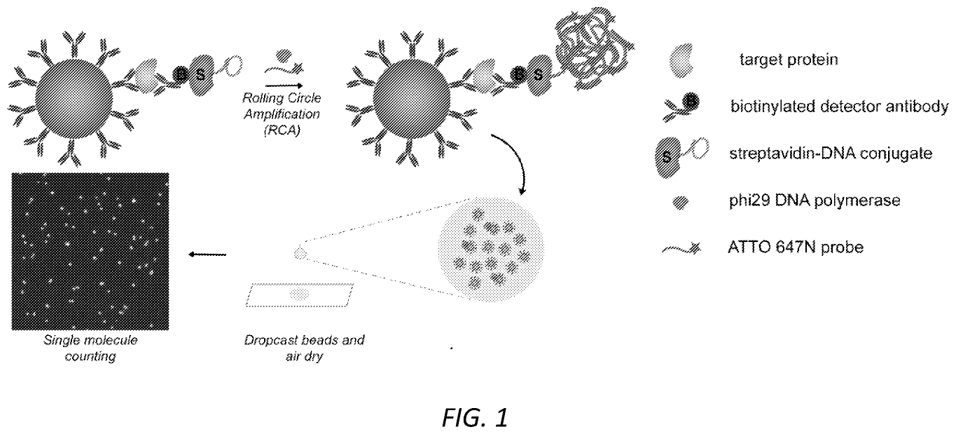

To enable bead dropcasting for counting of “on” and “off” beads, we first developed a strategy for generating a localized signal on each bead carrying a full immunocomplex sandwich. Rolling circle amplification (RCA), an isothermal DNA amplification method based on the processive action of a polymerase around a circular DNA template, generates long concatemers of DNA repeats to provide rapid and strong signal amplification. As RCA has been successfully used for the detection of individual protein-protein complexes and nucleic acids, we hypothesized that RCA would enable detection of single immunocomplex sandwiches captured on beads.²⁰⁻²³ RCA has also been performed on immunocomplexes on beads isolated in microwell arrays to enable multiplexed protein detection.²⁴ To incorporate RCA into our single molecule detection platform, we labeled each immunocomplex sandwich with an RCA primer annealed with a circular DNA template (FIG. 1). After RCA is performed, the generated DNA concatemer attached to each immunocomplex can be hybridized with a large number of complementary fluorescently labeled DNA probes for visualization.

The dSimoa method utilizes the same target capture steps as conventional Simoa, in which antibody-coated paramagnetic beads are incubated with the sample and biotinylated detector antibody to form an immunocomplex sandwich. However, instead of labeling the immunocomplex sandwich with streptavidin-β-galactosidase (SβG), streptavidin conjugated to a pre-annealed primer-template pair is used to label the immunocomplex sandwich. RCA is then carried out on each labeled immunocomplex sandwich at 37° C. for signal amplification. Furthermore, a fluorescently labeled DNA probe is added into the RCA reaction for in situ hybridization. After the RCA reaction, the beads are washed, concentrated, dropcast onto a microscope slide, and allowed to dry to form a monolayer film for imaging. As our preliminary attempts of directly using a detector antibody-DNA conjugate for immunocomplex formation followed by RCA resulted in high background signals (data not shown), we used a streptavidin-DNA conjugate for all dSimoa assays.

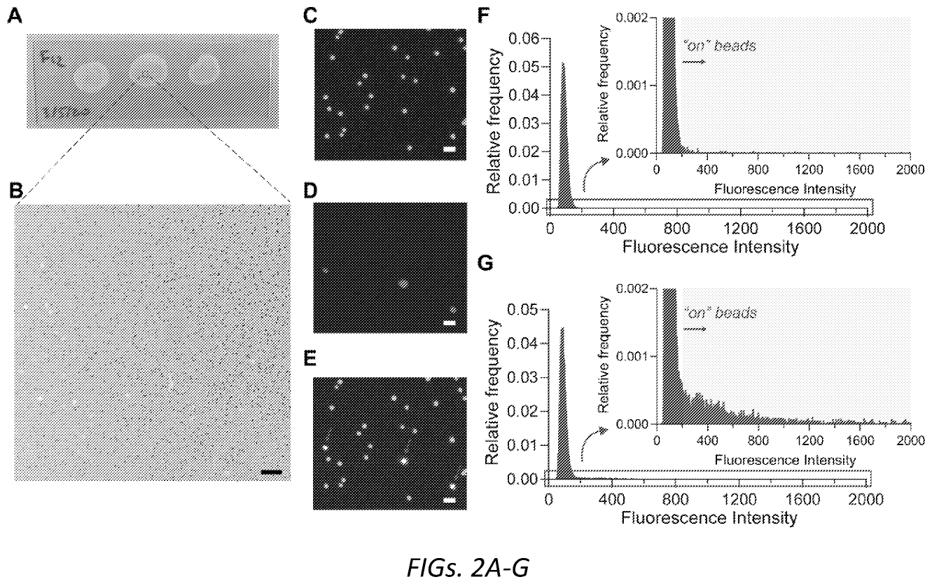

To evaluate the signal amplification and bead distribution in the dropcast films, we used dSimoa to detect interleukin-1 beta (IL-1β) as a model analyte, with the same capture and detector antibody pair used in a previously validated Simoa assay. Fluorescent dye-encoded beads (488 nm) were used to facilitate bead identification in the dropcast film for analysis, as salt crystal formation from the dropcast buffer could interfere with bead identification in brightfield images. With 100,000 assay beads and a dropcast volume of approximately 15 μL, the dropcast bead films show minimal bead aggregation and high, uniform bead densities across the film (FIGS. 2A-B). Furthermore, the dropcast process is rapid, with 15 dropcast volumes drying into films of 12-15 mm diameter within fifteen minutes. The presence of a captured target analyte on a bead is indicated by a fluorescent signal covering all or part of a bead (FIGS. 2C-E). As inclusion of aggregated beads in image analysis can affect the accuracy of the calculated fraction of “on” beads, bead aggregates of two or more beads, which constituted about 20-25% of the beads in each film, were separated by watershed segmentation in the image analysis algorithm, and any remaining bead aggregates were excluded from analysis via a size threshold. Representative histograms of the maximum fluorescence intensities on all imaged beads in the dropcast film show a wide range of “on” bead signal intensities, due to the broad size distribution of concatemers generated by RCA (FIGS. 2F-G). The number of “on” and “off” beads in each dropcast film was calculated by fitting a normal distribution to the maximum fluorescence intensities of each bead and assigning a threshold for “on” beads as five standard deviations above the mean. The average target molecule per bead (AMB), analogous to the average enzyme per bead (AEB) calculated in conventional Simoa, was determined using the Poisson distribution equation.²⁵

By simply transferring the entire volume of beads to a microscope slide, we are able to image and analyze 40-50% of the total number of assay beads on average, with most of the remaining beads either lost during wash or transfer steps or excluded from analysis due to aggregate formation. Thus, the sampling efficiency in dSimoa represents a significant improvement over the ˜5% of beads analyzed using the current Simoa technology. In addition to eliminating the requirement for microwells, dSimoa also enables far fewer beads to be used for target capture due to the increased percentage of beads that can be analyzed, thus improving sampling of rare target molecules while minimizing Poisson noise. The current Simoa technology uses 500,000 beads, while dSimoa uses 100,000 beads. Decreasing the number of beads can increase the signal to background, as there will be more “on” beads relative to the total number of beads, and thereby a higher AMB. Furthermore, the fluorescent signal remains highly stable in the dropcast film in its dry state, with no decreases in measured AMB values even after one month (Table 2).

Digital Detection of Proteins with dSimoa

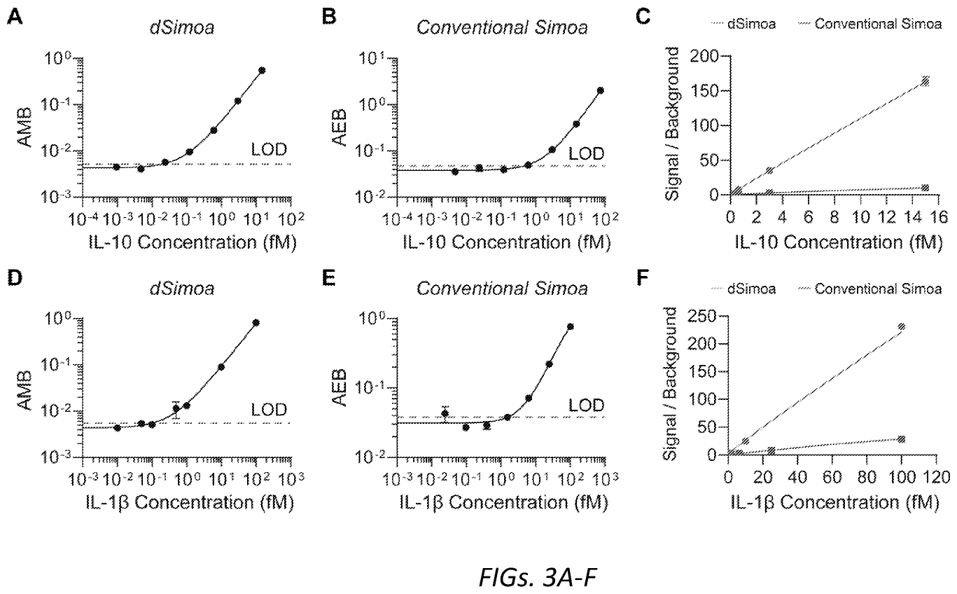

To assess the sensitivity of dSimoa, we generated calibration curves for two human cytokines, IL-1(3 and interleukin-10 (IL-10), using the same antibody pairs previously used in the corresponding Simoa assays. These dSimoa assays attained low- to mid-attomolar limits of detection (LODs), showing 25- and 15-fold improvements in sensitivity over the corresponding conventional Simoa assays for IL-10 and IL-1(3, respectively (FIGS. 3A-F; Table 1). The limits of quantification (LOQs), calculated as ten standard deviations above the background (AMB or AEB of the blank), were also improved by an order of magnitude in the dSimoa assays compared to the conventional Simoa assays. By substantially increasing the percentage of beads that can be analyzed, dSimoa enhances sampling efficiencies of low abundance molecules and enables far fewer beads to be used. In addition, the five-fold reduction in the number of beads increased the signal to background, contributing to the significant enhancements in sensitivity (FIG. 3C, F).

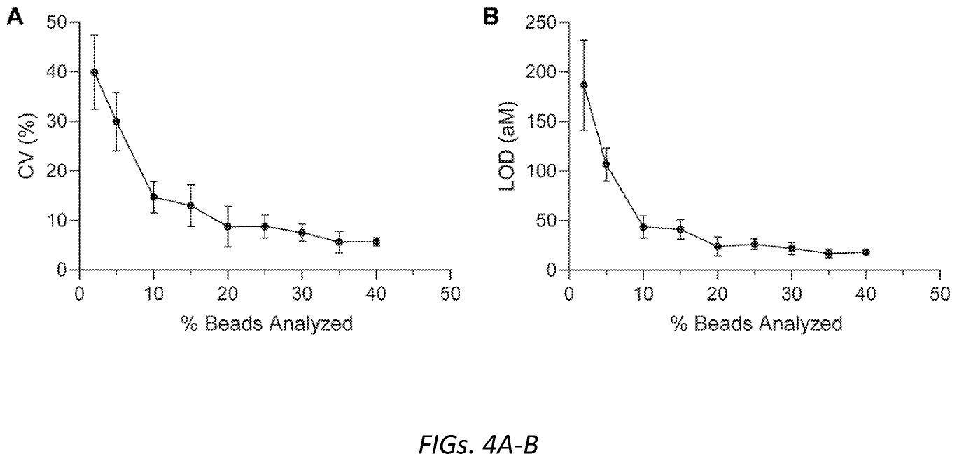

In addition to improving the signal to background, we hypothesized that increasing the sampling efficiencies of the captured target molecules also helped to achieve lower LODs with dSimoa, by reducing measurement imprecision due to Poisson noise. To determine whether our experimental results supported this hypothesis, we randomly selected subsets of the beads analyzed in the IL-10 calibration curve and determined the LODs and coefficients of variation (CV) of the background measurements at varying percentages of total assay beads analyzed (FIGS. 4A-B). When few beads (below 10% of the total beads) were analyzed, the imprecision of the background measurements was very high, with CVs of greater than 20%, which corresponded to poorer LODs, as expected from Poisson sampling noise. Moreover, there were high variations in the obtained LODs among different random samplings when low percentages of beads were analyzed. These observations thus demonstrate the important role of sampling efficiency in the precision and sensitivity of digital measurements. The calculated LOD values did not increase much further when 20% or more of the beads were analyzed, suggesting that close to maximal sensitivity can be attained upon imaging at least 20% of the assay beads.

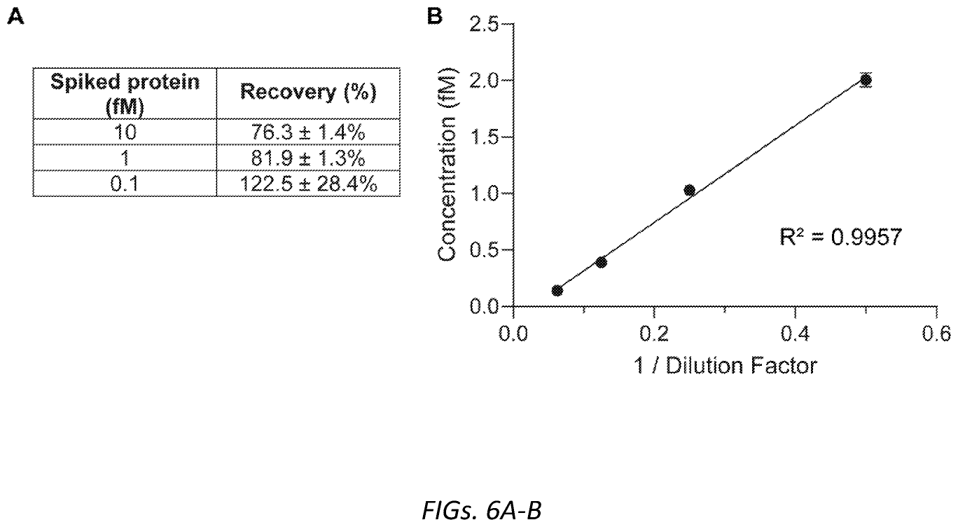

To validate the performance of dSimoa in biological fluids, we performed spike and recovery experiments in human saliva for IL-10. Recovery rates of various concentrations of recombinant human IL-10 protein spiked into pooled human saliva ranged from 76% to 122%, thus demonstrating that dSimoa can reliably detect proteins in saliva (FIG. 6A). Furthermore, dSimoa measurements of IL-10 in serial dilutions of human saliva showed linear dilution, indicating minimal interference from the saliva matrix on the dSimoa assay (FIG. 6B). The dSimoa assay also showed high measurement precision, with CVs well below 10% across all the saliva samples.

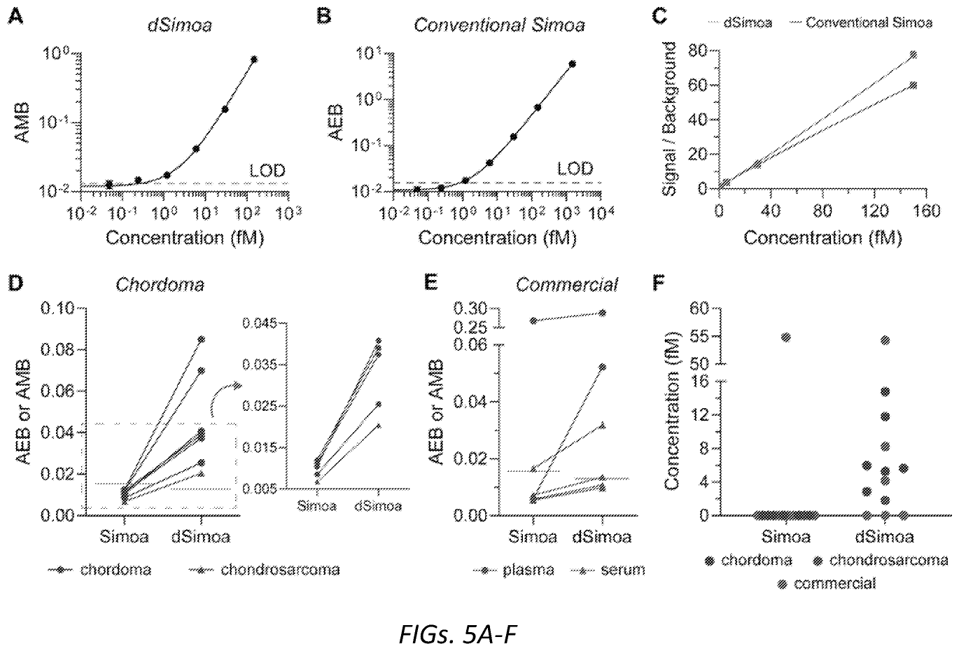

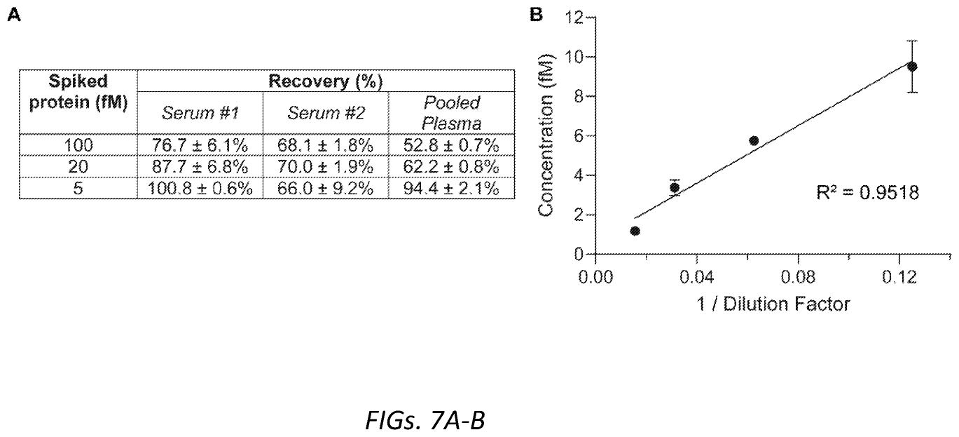

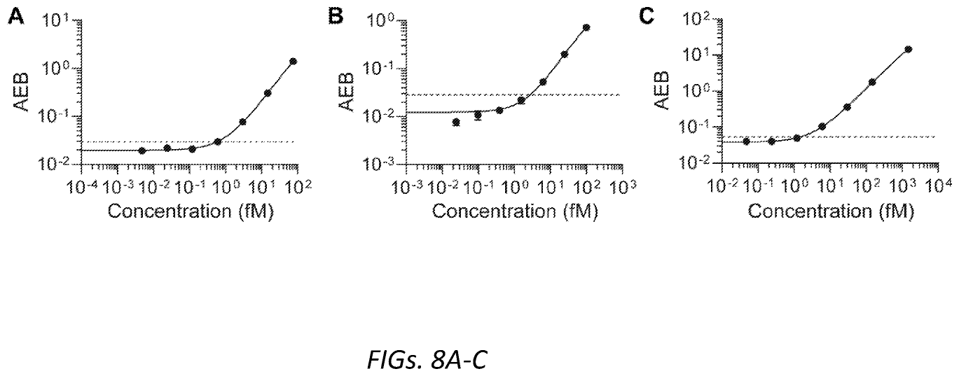

To explore the potential diagnostic utility of the improved sensitivity of dSimoa, we developed a dSimoa assay for Brachyury, a T-box transcription factor that is strongly linked to chordoma, a primary bone cancer in the spine or skull base.²⁶ While elevated levels of Brachyury expression have been found in chordoma tumors, there have been no reports on the measurement of Brachyury in plasma, to the best of our knowledge.²⁷⁻³⁰ The calibration curves generated by the dSimoa and conventional Simoa assays for Brachyury yielded LODs of 244.6 aM and 841.4 aM, respectively (FIGS. 5A-B). This dSimoa assay provides only a three-fold improvement in sensitivity over the conventional Simoa assay. The relatively small improvement in LOD may be attributed to the smaller increase in the signal to background compared to the conventional Simoa assay (FIG. 5C). As reducing the number of total assay beads also reduces the capture antibody concentration, the extent of improvement in signal to background from decreasing the number of assay beads may be smaller for antibodies with lower binding affinities, which can lead to decreased capture efficiencies despite the higher ratio of target molecules to beads. To validate the performance of the dSimoa assay in plasma and serum matrices, we performed spike and recovery experiments, obtaining recoveries of at least 65-70% in the majority of the spiked plasma and serum samples, with the majority of measurement CVs below 10% (FIG. 7A). We further validated the accuracy of the dSimoa assay in plasma by confirming acceptable dilution linearity (FIG. 7B).

Finally, we compared the abilities of dSimoa and conventional Simoa to detect endogenous Brachyury in several chordoma patient plasma samples as well as commercial plasma and serum samples from healthy donors. While the conventional Simoa measurements fell below its LOD for all six chordoma patient samples, dSimoa was able to measure detectable levels of Brachyury in all six samples (FIG. 5D), demonstrating that even a relatively small improvement in sensitivity is sometimes sufficient to measure clinically-important biomarkers. We also tested one chondrosarcoma patient sample, which was undetectable by conventional Simoa but detectable by dSimoa. Among the six commercial plasma and serum samples, Brachyury was detectable in one sample using conventional Simoa and in three samples using dSimoa (FIG. 5E). Notably, although the measured concentrations by dSimoa in many of the samples were in the low femtomolar range, above the calculated LOD of the conventional Simoa assay, measurements of these samples by conventional Simoa still fell below its LOD (FIG. 5F). However, among the AEBs that were below the conventional Simoa LOD, higher AEB values generally correlated to higher AMB values and measured concentrations in the dSimoa assay. Furthermore, at higher sample concentrations, dSimoa and conventional Simoa yielded similar measured concentrations. The superior performance of dSimoa in plasma and serum at low concentrations may be attributed to several factors, including the improved LOD of dSimoa and better sampling efficiencies, which can increase measurement precision particularly at low concentrations. Another possibility is that dSimoa performs more accurately in plasma and serum matrices than conventional Simoa, with higher signal to background and recoveries in plasma and serum using five-fold fewer beads. In addition, conventional Simoa employs a large enzyme label that may exhibit higher non-specific binding than the much smaller oligonucleotide label used in dSimoa. Finally, the greater amount of washing during the dSimoa assay compared to the conventional Simoa assay may have further reduced interference from plasma and serum components.

TABLE 1
Calculated limits of detection (LODs) and limits of quantification
(LOQs) for IL-10 and IL-1β using dSimoa and conventional Simoa.

| | LOD | | | | | LOQ | |
|---|---|---|---|---|---|---|---|
| | | Conventional | | | | | Conventional |
| Target | dSimoa | Simoa | | Quanterix | | dSimoa | Simoa |
| IL-10 | 19.2 aM | 485.0 | aM | 204.3 | aM | 64.5 aM | 1.57 fM |
| IL-1β | 99.6 aM | 1.52 | fM | 941 | fM | 384.4 aM | 5.46 fM |

LOD and LOQ values were calculated as three and ten standard deviations above the background, respectively. The reported LOD values by the corresponding Quanterix Simoa assays were calculated as 2.5 standard deviations above the background.

TABLE 2
Signal stability in dropcast bead films over time.

| | AMB | |
|---|---|---|
| Days | Sample #1 | Sample #2 |
| 0 | 0.0095 | 0.0246 |
| 4 | 0.0094 | 0.0246 |
| 7 | 0.0106 | 0.0253 |
| 15 | 0.0093 | 0.0251 |
| 30 | 0.0104 | 0.0252 |

Dropcast films were imaged at various days post-formation and average molecule per bead (AMB) values were calculated.

TABLE 3
AMB and AEB values for calibration curves generated
by dSimoa and conventional Simoa, respectively.

| | dSimoa | | | | | | |
|---|---|---|---|---|---|---|---|
| | 300 pM | | | | | | |
| | streptavidin- | | Conventional Simoa | | | | |
| IL-1B | DNA/15 min | | IL-1B | 300 pM SβG/15 min | | 150 pM SβG/5 min | |
| Concentration | average | CV | Concentration | average | CV | average | CV |
| (fM) | AMB | (%) | (fM) | AEB | (%) | AEB | (%) |
| 0 | 0.0042 | 10.1 | 0 | 0.0269 | 13.6 | 0.0189 | 17.1 |
| 0.01 | 0.0044 | 11.7 | 0.024 | 0.0428 | 25.7 | 0.0078 | 17.4 |
| 0.05 | 0.0054 | 4.1 | 0.098 | 0.0271 | 3.9 | 0.0108 | 21.8 |
| 0.1 | 0.0051 | 10.6 | 0.391 | 0.0290 | 11.9 | 0.0135 | 10.8 |
| 0.5 | 0.0114 | 39.6 | 1.563 | 0.0379 | 9.0 | 0.0222 | 16.9 |
| 1 | 0.0133 | 9.4 | 6.25 | 0.0714 | 2.5 | 0.0528 | 5.7 |
| 10 | 0.0898 | 1.2 | 25 | 0.2212 | 2.7 | 0.1988 | 2.6 |
| 100 | 0.8218 | 1.5 | 100 | 0.7666 | 2.9 | 0.7212 | 2.5 |
| | 150 pM | | | | | | |
| | streptavidin- | | | | | | |
| IL-10 | DNA/15 min | | IL-10 | 150 pM SβG/15 min | | 150 pM SβG/5 min | |
| Concentration | average | CV | Concentration | average | CV | average | CV |
| (fM) | AMB | (%) | (fM) | AEB | (%) | AEB | (%) |
| 0 | 0.0044 | 6.5 | 0 | 0.0380 | 8.8 | 0.0190 | 18.6 |
| 0.00096 | 0.0045 | 12.8 | 0.0048 | 0.0355 | 6.3 | 0.0194 | 4.4 |
| 0.0048 | 0.0042 | 15.2 | 0.024 | 0.0439 | 16.4 | 0.0218 | 9.2 |
| 0.024 | 0.0058 | 6.7 | 0.12 | 0.0395 | 7.6 | 0.0208 | 12.9 |
| 0.12 | 0.0097 | 6.1 | 0.6 | 0.0500 | 9.5 | 0.0296 | 7.2 |
| 0.6 | 0.0281 | 2.1 | 3 | 0.1073 | 4.2 | 0.0765 | 0.5 |
| 3 | 0.1214 | 5.3 | 15 | 0.3858 | 3.1 | 0.3056 | 4.7 |
| 15 | 0.5615 | 3.6 | 75 | 2.0478 | 2.9 | 1.4073 | 2.7 |
| | 300 pM | | | | | | |
| | streptavidin- | | | | | | |
| Brachyury | DNA/15 min | | Brachyury | 300 pM SβG/15 min | | 150 pM SβG/5 min | |
| Concentration | average | CV | Concentration | average | CV | average | CV |
| (fM) | AMB | (%) | (fM) | AEB | (%) | AEB | (%) |
| 0 | 0.0106 | 7.8 | 0 | 0.0382 | 13.0 | 0.0113 | 12.8 |
| 0.048 | 0.0127 | 12.6 | 0.048 | 0.0398 | 8.6 | 0.0108 | 18.0 |
| 0.24 | 0.0147 | 9.3 | 0.24 | 0.0395 | 2.7 | 0.0120 | 9.8 |
| 1.2 | 0.0173 | 6.7 | 1.2 | 0.0494 | 2.0 | 0.0174 | 13.2 |
| 6 | 0.0417 | 9.0 | 6 | 0.1046 | 2.4 | 0.0425 | 9.0 |
| 30 | 0.1556 | 3.7 | 30 | 0.3583 | 4.1 | 0.1561 | 4.1 |
| 150 | 0.8248 | 2.7 | 150 | 1.7665 | 0.6 | 0.6767 | 3.0 |
| | | | 1500 | 14.3454 | 2.4 | 5.9720 | 2.9 |

TABLE 4
Summary of dSimoa assay conditions.

| | Detector | | |
|---|---|---|---|
| | Antibody | Streptavidin-DNA | |
| | Concentration | Concentration | Incubation Times |
| Target | (μg/mL) | (pM) | (antibody-streptavidin) |
| IL-1β | 0.6 | 300 | 60 min-15 min |
| IL-10 | 0.3 | 150 | 60 min-15 min |
| Brachyury | 0.1 | 300 | 60 min-15 min |

Conventional Simoa assays were performed using the same detector antibody concentrations and incubation times, including an assay with the same SβG concentrations and incubation times and an assay with standard SβG conditions from previously developed assays (150 pM SβG for five minutes).

TABLE 5
Antibodies and recombinant protein standards used in dSimoa and
conventional Simoa assays.

| Reagent | Manufacturer |
|---|---|
| IL-1β capture antibody | Biolegend 508202 |
| IL-1β detector antibody | Biolegend 511703 |
| IL-1β protein standard | R&D Systems 201-LB-005 |
| IL-10 capture antibody | Biolegend 506802 |
| IL-10 detector antibody | R&D Systems 217-IL-005 |
| IL-10 protein standard | Biolegend 501501 |
| Brachyury capture antibody | Abcam ab236023 |
| Brachyury detector antibody | R&D Systems BAF2085 |
| Brachyury protein standard | Abcam ab114235 |

TABLE 6

| DNA sequences used in dSimoa assays. | |
|---|---|
| | Sequence |
| Primer | 5′-Azide-TTTTTTTTTTTTTTTTAGACACCGTTCCTTG |
| | GACAGA*G*C (SEQ ID NO: 1) |
| Template | 5′-Phosphate-GAACGGTGTCTATTATGTCCTATCCTC |
| | AGCTATTATGTCCTATCCTCAGC TATTATGTCCTATCCT |
| | CAGCTCTGTCCAAG (SEQ ID NO: 2) |
| Probe | 5′-ATTO 647N-TATTATGTCCTATCCTCAGC-InvdT |
| | (SEQ ID NO: 3) |

Bolded regions correspond to the complementary regions in the RCA template and primer.

### REFERENCES FOR EXAMPLE 1

1. Cohen, L.; Walt, D. R., Highly Sensitive and Multiplexed Protein Measurements. Chemical Reviews 2019, 119 (1), 293-321.
2. Rissin, D. M.; Kan, C. W.; Campbell, T. G.; Howes, S. C.; Fournier, D. R.; Song, L.; Piech, T.; Patel, P. P.; Chang, L.; Rivnak, A. J.; Ferrell, E. P.; Randall, J. D.; Provuncher, G. K.; Walt, D. R.; Duffy, D. C., Single-molecule enzyme-linked immunosorbent assay detects serum proteins at subfemtomolar concentrations. Nature Biotechnology 2010, 28, 595.
3. Rissin, D. M.; Walt, D. R., Digital Readout of Target Binding with Attomole Detection Limits via Enzyme Amplification in Femtoliter Arrays. Journal of the American Chemical Society 2006, 128 (19), 6286-6287.
4. Rissin, D. M.; Walt, D. R., Digital Concentration Readout of Single Enzyme Molecules Using Femtoliter Arrays and Poisson Statistics. Nano Letters 2006, 6 (3),
5. Yelleswarapu, V.; Buser, J. R.; Haber, M.; Baron, J.; Inapuri, E.; Issadore, D., Mobile platform for rapid sub—picogram-per-milliliter, multiplexed, digital droplet detection of proteins. Proceedings of the National Academy of Sciences 2019, 116 (10), 4489.
6. Robey, T. T.; Panegyres, P. K., Cerebrospinal fluid biomarkers in neurodegenerative disorders. Future Neurology 2019, 14 (1), FNL6.
7. Olsson, B.; Lautner, R.; Andreasson, U.; Öhrfelt, A.; Portelius, E.; Bjerke, M.; Hölttä, M.; Rosén, C.; Olsson, C.; Strobel, G.; Wu, E.; Dakin, K.; Petzold, M.; Blennow, K.; Zetterberg, H., CSF and blood biomarkers for the diagnosis of Alzheimer's disease: a systematic review and meta-analysis. The Lancet Neurology 2016, 15 (7), 673-684.
8. Galasko, D. R.; Shaw, L. M., CSF biomarkers for Alzheimer disease—approaching consensus. Nature Reviews Neurology 2017, 13 (3), 131-132.
9. Fortea, J.; Carmona-Iragui, M.; Benejam, B.; Fernândez, S.; Videla, L.; Barroeta, I.; Alcolea, D.; Pegueroles, J.; Muñoz, L.; Belbin, 0.; de Leon, M. J.; Maceski, A. M.; Hirtz, C.; Clarimón, J.; Videla, S.; Delaby, C.; Lehmann, S.; Blesa, R.; Lleó, A., Plasma and CSF biomarkers for the diagnosis of Alzheimer's disease in adults with Down syndrome: a cross-sectional study. The Lancet Neurology 2018, 17 (10), 860-869.
10. Hampel, H.; O'Bryant, S. E.; Molinuevo, J. L.; Zetterberg, H.; Masters, C. L.; Lista, S.; Kiddle, S. J.; Batrla, R.; Blennow, K., Blood-based biomarkers for Alzheimer disease: mapping the road to the clinic. Nature Reviews Neurology 2018, 14 (11), 639-652.
11. Simrén, J.; Ashton, N. J.; Blennow, K.; Zetterberg, H., An update on fluid biomarkers for neurodegenerative diseases: recent success and challenges ahead. Current Opinion in Neurobiology 2020, 61, 29-39.
12. Parnetti, L.; Gaetani, L.; Eusebi, P.; Paciotti, S.; Hansson, 0.; El-Agnaf, 0.; Mollenhauer, B.; Blennow, K.; Calabresi, P., CSF and blood biomarkers for Parkinson's disease. The Lancet Neurology 2019, 18 (6), 573-586.
13. Cohen, L.; Cui, N.; Cai, Y.; Garden, P. M.; Li, X.; Weitz, D. A.; Walt, D. R., Single Molecule Protein Detection with Attomolar Sensitivity using Droplet Digital ELISA. Under revision.
14. Kim, S. H.; Iwai, S.; Araki, S.; Sakakihara, S.; Iino, R.; Noji, H., Large-scale femtoliter droplet array for digital counting of single biomolecules. Lab on a Chip 2012, 12 (23), 4986-4991.
15. Witters, D.; Knez, K.; Ceyssens, F.; Puers, R.; Lammertyn, J., Digital microfluidics-enabled single-molecule detection by printing and sealing single magnetic beads in femtoliter droplets. Lab on a Chip 2013, 13 (11), 2047-2054.
16. Wilson, D. H.; Rissin, D. M.; Kan, C. W.; Fournier, D. R.; Piech, T.; Campbell, T. G.; Meyer, R. E.; Fishburn, M. W.; Cabrera, C.; Patel, P. P.; Frew, E.; Chen, Y.; Chang, L.; Ferrell, E. P.; von Einem, V.; McGuigan, W.; Reinhardt, M.; Sayer, H.; Vielsack, C.; Duffy, D. C., The Simoa HD-1 Analyzer: A Novel Fully Automated Digital Immunoassay Analyzer with Single-Molecule Sensitivity and Multiplexing. Journal of Laboratory Automation 2015, 21 (4), 533-547.
17. Barbee, K. D.; Hsiao, A. P.; Heller, M. J.; Huang, X., Electric field directed assembly of high-density microbead arrays. Lab on a Chip 2009, 9 (22), 3268-3274.
18. Decrop, D.; Pardon, G.; Brancato, L.; Kil, D.; Zandi Shafagh, R.; Kokalj, T.; Haraldsson, T.; Puers, R.; van der Wijngaart, W.; Lammertyn, J., Single-Step Imprinting of Femtoliter Microwell Arrays Allows Digital Bioassays with Attomolar Limit of Detection. ACS Applied Materials & Interfaces 2017, 9 (12), 10418-10426.
19. Decrop, D.; Brans, T.; Gijsenbergh, P.; Lu, J.; Spasic, D.; Kokalj, T.; Beunis, F.; Goos, P.; Puers, R.; Lammertyn, J., Optical Manipulation of Single Magnetic Beads in a Microwell Array on a Digital Microfluidic Chip. Analytical Chemistry 2016, 88 (17), 8596-8603.
20. Jarvius, J.; Melin, J.; Göransson, J.; Stenberg, J.; Fredriksson, S.; Gonzalez-Rey, C.; Bertilsson, S.; Nilsson, M., Digital quantification using amplified single-molecule detection. Nature Methods 2006, 3 (9), 725-727.
21. Söderberg, 0.; Gullberg, M.; Jarvius, M.; Ridderstråle, K.; Leuchowius, K.-J.; Jarvius, J.; Wester, K.; Hydbring, P.; Bahram, F.; Larsson, L.-G.; Landegren, U., Direct observation of individual endogenous protein complexes in situ by proximity ligation. Nature Methods 2006, 3 (12), 995-1000.
22. Kühnemund, M.; Hernández-Neuta, I.; Sharif, Mohd I.; Cornaglia, M.; Gijs, M. A. M.; Nilsson, M., Sensitive and inexpensive digital DNA analysis by microfluidic enrichment of rolling circle amplified single-molecules. Nucleic Acids Research 2017, 45 (8), e59-e59.
23. Lizardi, P. M.; Huang, X.; Zhu, Z.; Bray-Ward, P.; Thomas, D. C.; Ward, D. C., Mutation detection and single-molecule counting using isothermal rolling-circle amplification. Nature Genetics 1998, 19 (3), 225-232.
24. Konry, T.; Hayman, R. B.; Walt, D. R., Microsphere-Based Rolling Circle Amplification Microarray for the Detection of DNA and Proteins in a Single Assay. Analytical Chemistry 2009, 81 (14), 5777-5782.
25. Rissin, D. M.; Fournier, D. R.; Piech, T.; Kan, C. W.; Campbell, T. G.; Song, L.; Chang, L.; Rivnak, A. J.; Patel, P. P.; Provuncher, G. K.; Ferrell, E. P.; Howes, S. C.; Pink, B. A.; Minnehan, K. A.; Wilson, D. H.; Duffy, D. C., Simultaneous Detection of Single Molecules and Singulated Ensembles of Molecules Enables Immunoassays with Broad Dynamic Range. Analytical Chemistry 2011, 83 (6), 2279-2285.
26. Vujovic, S.; Henderson, S.; Presneau, N.; Odell, E.; Jacques, T. S.; Tirabosco, R.; Boshoff, C.; Flanagan, A. M., Brachyury, a crucial regulator of notochordal development, is a novel biomarker for chordomas. The Journal of Pathology 2006, 209 (2), 157-165.
27. Presneau, N.; Shalaby, A.; Ye, H.; Pillay, N.; Halai, D.; Idowu, B.; Tirabosco, R.; Whitwell, D.; Jacques, T. S.; Kindblom, L.-G.; Brüderlein, S.; Möller, P.; Leithner, A.; Liegl, B.; Amary, F. M.; Athanasou, N. N.; Hogendoorn, P. C. W.; Mertens, F.; Szuhai, K.; Flanagan, A. M., Role of the transcription factor T (brachyury) in the pathogenesis of sporadic chordoma: a genetic and functional-based study. The Journal of Pathology 2011, 223 (3), 327-335.
28. Yang, X. R.; Ng, D.; Alcorta, D. A.; Liebsch, N. J.; Sheridan, E.; Li, S.; Goldstein, A. M.; Parry, D. M.; Kelley, M. J., T (brachyury) gene duplication confers major susceptibility to familial chordoma. Nature Genetics 2009, 41 (11), 1176-1178.
29. Shen, J.; Li, C.-D.; Yang, H.-L.; Lu, J.; Zou, T.-M.; Wang, D.-L.; Deng, M., Classic chordoma coexisting with benign notochordal cell rest demonstrating different immunohistological expression patterns of brachyury and galectin-3. Journal of Clinical Neuroscience 2011, 18 (1), 96-99.
30. Miettinen, M.; Wang, Z.; Lasota, J.; Heery, C.; Schlom, J.; Palena, C., Nuclear Brachyury Expression Is Consistent in Chordoma, Common in Germ Cell Tumors and Small Cell Carcinomas, and Rare in Other Carcinomas and Sarcomas: An Immunohistochemical Study of 5229 Cases. The American Journal of Surgical Pathology 2015, 39 (10), 1305-1312.
31. Walcott, B. P.; Nahed, B. V.; Mohyeldin, A.; Coumans, J.-V.; Kahle, K. T.; Ferreira, M. J., Chordoma: current concepts, management, and future directions. The Lancet Oncology 2012, 13 (2), e69-e76.
32. Bergh, P.; Kindblom, L.-G.; Gunterberg, B.; Remotti, F.; Ryd, W.; Meis-Kindblom, J. M., Prognostic factors in chordoma of the sacrum and mobile spine. Cancer 2000, 88 (9), 2122-2134.
33. Boyd-Moss, M.; Baratchi, S.; Di Venere, M.; Khoshmanesh, K., Self-contained microfluidic systems: a review. Lab on a Chip 2016, 16 (17), 3177-3192.
34. Zhang, Y.; Nguyen, N.-T., Magnetic digital microfluidics—a review. Lab on a Chip 2017, 17 (6), 994-1008.
35. Nie, S.; Henley, W. H.; Miller, S. E.; Zhang, H.; Mayer, K. M.; Dennis, P. J.; Oblath, E. A.; Alarie, J. P.; Wu, Y.; Oppenheim, F. G.; Little, F. F.; Uluer, A. Z.; Wang, P.; Ramsey, J. M.; Walt, D. R., An automated integrated platform for rapid and sensitive multiplexed protein profiling using human saliva samples. Lab on a Chip 2014, 14 (6), 1087-1098.
36. Sista, R. S.; Eckhardt, A. E.; Srinivasan, V.; Pollack, M. G.; Palanki, S.; Pamula, V. K., Heterogeneous immunoassays using magnetic beads on a digital microfluidic platform. Lab on a Chip 2008, 8 (12), 2188-2196.
37. Wei, Q.; Qi, H.; Luo, W.; Tseng, D.; Ki, S. J.; Wan, Z.; Gorocs, Z.; Bentolila, L. A.; Wu, T.-T.; Sun, R.; Ozcan, A., Fluorescent Imaging of Single Nanoparticles and Viruses on a Smart Phone. ACS Nano 2013, 7 (10), 9147-9155.
38. Kühnemund, M.; Wei, Q.; Darai, E.; Wang, Y.; Hernandez-Neuta, I.; Yang, Z.; Tseng, D.; Ahlford, A.; Mathot, L.; Sjöblom, T.; Ozcan, A.; Nilsson, M., Targeted DNA sequencing and in situ mutation analysis using mobile phone microscopy. Nature Communications 2017, 8, 13913.
39. Kong, J. E.; Wei, Q.; Tseng, D.; Zhang, J.; Pan, E.; Lewinski, M.; Garner, O. B.; Ozcan, A.; Di Carlo, D., Highly Stable and Sensitive Nucleic Acid Amplification and Cell-Phone-Based Readout. ACS Nano 2017, 11 (3), 2934-2943.
40. Minagawa, Y.; Ueno, H.; Tabata, K. V.; Noji, H., Mobile imaging platform for digital influenza virus counting. Lab on a Chip 2019, 19 (16), 2678-2687.
41. Ghosh, K. K.; Burns, L. D.; Cocker, E. D.; Nimmerjahn, A.; Ziv, Y.; Gamal, A. E.; Schnitzer, M. J., Miniaturized integration of a fluorescence microscope. Nature Methods 2011, 8 (10), 871-878.
42. Cohen, L.; Hartman, M. R.; Amardey-Wellington, A.; Walt, D. R., Digital direct detection of microRNAs using single molecule arrays. Nucleic Acids Research 2017, 45 (14), e137-e137.
43. Wang, X.; Cohen, L.; Wang, J.; Walt, D. R., Competitive Immunoassays for the Detection of Small Molecules Using Single Molecule Arrays. Journal of the American Chemical Society 2018, 140 (51), 18132-18139.

### Example 2. Simplified Digital Enzyme-linked Immunosorbent Assay Using Tyramide Signal Amplification and Fibrin Hydrogels

Methods

Materials. All materials were received and used per manufacturer's instructions unless otherwise specified below. IL-6 protein standard (#206-IL-010) and antibodies (capture #MAB206 and detection #BAF206) were purchased from R&D Systems.

Preparation of antibody-coated capture beads. IL-6 antibody was first buffer exchanged to remove storage buffer. 0.13 mg of antibody was added to a 50 K Amicon Ultra-0.5 mL Centrifugal Filter (MilliporeSigma). Bead Conjugation Buffer (Quanterix Corp.) was added to the filter to a volume of 500 μL. The filter device was centrifuged at 14,000×g for five minutes. After centrifugation, the effluent was discarded and additional Bead Conjugation Buffer was added to the filter (total volume of 500 μL). The centrifugation process was repeated twice more. The filter was then inverted into a new tube and centrifuged at 1,000×g for two minutes. The concentration of the collected antibody was measured using a NanoDrop One (ThermoFisher). The buffer-exchanged antibody was diluted to 0.5 mg/mL using Bead Conjugation Buffer. To prepare beads for conjugation, 2.8×10⁸ Quanterix 647 nm dye-encoded carboxylated paramagnetic beads (2.7 μm) were washed three times with Bead Wash Buffer (Quanterix), three times with Bead Conjugation Buffer, and then resuspended in 190 μL of Bead Conjugation Buffer. Prior to use, 1 mg of 1-ethyl-3-(3-dimethylaminopropyl)carbodiimide hydrochloride (EDC) was dissolved in 100 μL of Bead Conjugation Buffer. After the beads were washed, 10 μL of EDC was added to the beads and the beads were agitated on a rotator for 30 min. After bead activation with EDC, the beads were washed once with Bead Conjugation Buffer, and then resuspended in 200 μL of 0.5 mg/mL capture antibody solution. The beads were agitated on the rotator for 2 hours. After conjugation, the beads were washed two times with Bead Wash Buffer and then blocked with BSA for 30 min. in 200 μL of Bead Blocking Buffer (Quanterix). Finally, the antibody-conjugated beads were wash with Bead Wash Buffer, Bead Diluent (Quanterix), and resuspended in 200 μL of Bead Diluent. Beads were counted using a Beckman Coulter Z1 Particle Counter and stored at 4° C.

CARD digital ELISA. To generate calibration curves using CARD-dELISA, a three-step assay was performed to capture and label target proteins on beads and perform the on-bead signal amplification step. In the first step, IL-6 protein standard was serially diluted in Homebrew Sample Diluent (Quanterix) and 100 μL of each calibration standard was added to a low-bind 96-well plate (Greiner Bio-One). IL-6 capture beads (10 μL at 20,000 beads/μL) and 10 μL of biotinylated IL-6 detection antibody (final concentration 0.3 μg/mL) were also added to the 96-well plate. The plate was incubated with shaking for 1 hour and then washed 3 times with System Wash Buffer 1 (Quanterix). After the final wash cycle, the residual wash buffer was removed and the beads were resuspended in 100 μL of Sample Diluent. In the second step, 10 μL of 5 μg/mL streptavidin-poly-HRP (Thermo Scientific Pierce) was added to each sample. The plate was incubated with shaking for 10 min. and then washed 3 times with System Wash Buffer 1. After the final wash cycle, the residual wash buffer was removed. In the third step, the on-bead tyramide signal amplification was performed using the Alexa Fluor 488 Tyramide SuperBoost Kit (ThermoFisher Scientific) with a modified protocol. Specifically, the working solution was prepared by mixing 1.6 mL of 1X reaction buffer with 16 μL of 1X hydrogen peroxide solution and 16 μL of Alexa Fluor 488-tyramide reagent. Then, beads were resuspended in 200 of the tyramide working solution. The plate was incubated with no shaking for 1 hour. After labeling, 50 μL of 1:11 diluted Reaction Stop Solution (SuperBoost Kit) was added to each bead suspension and incubated with shaking for 2 min. The plate was then washed 6 times with System Wash Buffer 1 with 1:11 diluted Reaction Stop Solution. After the final wash cycle, beads were resuspended in 30 μL of lx PBS. Beads were then added to silicon isolation wells (Electron Microscopy Sciences) on a glass microscope slide. To prepare the fibrin hydrogel, equal parts of a 10 mg/mL solution of fibrinogen (from bovine plasma, Type I-S, MilliporeSigma) in 1×PBS and a 1.25 U/mL solution of thrombin (from bovine plasma, MilliporeSigma) in 1×PBS were mixed. In order for fibrinogen to dissolve in PBS, solutions were heated at 37° C. prior to use. After mixing the hydrogel reagents, 50 μL of the mixture was added to each isolation well and the hydrogel was allowed to form for 15 minutes before imaging.

Imaging and Analysis. Brightfield and fluorescent images of the hydrogel immobilized bead arrays were acquired with an Olympus IX81 inverted microscope at 10× magnification with an OCRA-Flash 4.0 LT+CMOS camera (Hamamatsu). CellSens software was used to control the microscope stage and acquire images. Brightfield images, which were used to identify the location of beads, were acquired with an exposure time of 20 ms. Fluorescence images using a GFP filter cube, which were used to identify “on” and “off” beads, were acquired with an exposure time of 1 s.

Image analysis was performed using a custom MATLAB algorithm. Brightfield images were processed by computing the complement of the image (so that beads were bright and the background was dark), filtering with a top-hat filter to correct for uneven illumination, and converting to a binary image. Fluorescent images were also filtered with a top-hat filter to correct for uneven illumination. Beads were located in brightfield images using a disk-shaped morphological structuring element. The signal intensity of each bead was measured from corresponding GFP fluorescent images by calculating the intensity in the top quartile in the bead region. The cut-off value between “off” and “on” beads was determine by fitting the distribution of bead intensities in the blank (0 fM) standard to a normal distribution and setting the cut-off intensity to three standard deviations above the mean for low concentration samples (<50 fM, cut-off value of −90) and four standard deviations above the mean for high concentration samples (≥50 fM, cut-off value ˜100). The fraction of “on” beads was calculated by dividing the number of “on” beads by the total number of beads.

Calibration curves of AEB vs. concentration were fit to a four-parameter logistic (4PL) regression in GraphPad Prism version 8.3.0. The 4PL fit curves were used to determine unknown IL-6 concentrations in saliva samples. All measurements were performed in duplicate.

Saliva Sample Analysis. Pooled saliva samples were purchased from BioIVT and stored at −80° C. until ready for use. Saliva was centrifuged at 13,150×g for 20 min. at 4° C. The supernatant was removed after centrifugation and saliva samples were diluted 25X for CARD-dELISA analysis and 8X for Simoa analysis.

Simoa Assays. Conventional Simoa assays were performed on an HD-1 Analyzer (Quanterix). Solutions of capture beads, detection antibodies, and streptavidin-β-galactosidase (513G) were placed in reagent bottles and loaded onto the instrument. Serially diluted IL-6 protein standard for calibration curves and diluted saliva samples were pipetted into a 96 well plate (Quanterix) and loaded onto the instrument. SβG substrate resorufin β-D-galactopyranoside, System Wash Buffer 1, System Wash Buffer 2, and Simoa Sealing Oil were received from Quanterix and loaded onto the instrument following manufacturer's instructions. Standards and samples were processed using a standard three-step assay. Image analyses and AEB calculations were performed automatically by the on-board Simoa software.

Results

We sought to develop a simplified digital ELISA format that would reduce the need for expensive equipment and sophisticated microfluidics or robotics. By implementing a signal amplification step where fluorophores are conjugated directly to the beads, we eliminate the need for compartmentalization of beads in microwell arrays or microfluidic droplets. Instead, the signal amplification step occurs in bulk solution, such as in the same reaction chamber as the target protein capture and labeling steps. Furthermore, this approach doesn't require the expensive engineering and robotics that are required for bead loading into microwell arrays. CARD-dELISA has a simplified approach to bead imaging, which is used for single molecule counting, by immobilizing beads in a low-cost fibrin hydrogel. In this format, beads are arrayed on a glass slide and immobilized in the fibrin hydrogel layer as it forms in situ. After image acquisition, an algorithm in MATLAB is used to locate beads and measure their fluorescence intensity for single molecule counting. As a proof of concept, we generated a calibration curve for interleukin 6 (IL-6) and measured IL-6 levels in saliva samples.

The first step of developing CARD-dELISA was to establish and optimize a method for on-bead enzyme amplification for single-molecule signal generation. We use TSA, which is commonly used in immunohistochemistry and in situ hybridization experiments. Other researchers have also reported on the use of TSA for digital ELISA.²⁷ We improved on previous literature reports by reducing the number of steps in the immunoassay (reduced from a five-step assay to a three-step assay), which is an important consideration when implementing digital ELISA into point-of-care devices. The assay format of CARD-dELISA is summarized in FIG. 9A. Antibody-coated capture beads (200,000) are added to a sample to enable capture of target protein molecules. Similar to conventional Simoa, we use a large number of beads compared to the number of target protein molecules. This ensures that the assay follows the Poisson distribution, where most beads bind no target molecules and only a small percentage of beads bind one target molecule. After protein capture, the target molecule is labeled with a biotinylated detection antibody and streptavidin-poly-HRP (streptavidin-conjugated polymer with several horseradish peroxidase molecules), forming a full enzyme-labeled immunocomplex. The beads are then resuspended in a solution containing hydrogen peroxide and the tyramide-fluorophore conjugate (tyramide-Alexa Fluor 488) for the signal generation step. In the presence of hydrogen peroxide, HRP catalytically converts tyramide into a radical intermediate. This tyramide radical forms a covalent bond with other aromatic rings near the HRP molecules, such as tyrosine residues on nearby proteins and antibodies on the bead. At the completion of this step, beads with a full immunocomplex are labeled with a large number of covalently attached fluorescent dyes. This completes the on-bead signal generation step and allows for subsequent single molecule counting. We observe no detectable cross-labeling between beads after the tyramide labeling step. This is supported by the observation that only a small fraction of beads has detectable fluorescent signal at low protein concentrations, which is expected in this assay format that follows the Poisson distribution. Furthermore, in the tyramide labeling step, we use dilute bead solutions such that it is unlikely for a tyramide radical to diffuse to another bead during the lifetime of the radical intermediate.²⁷

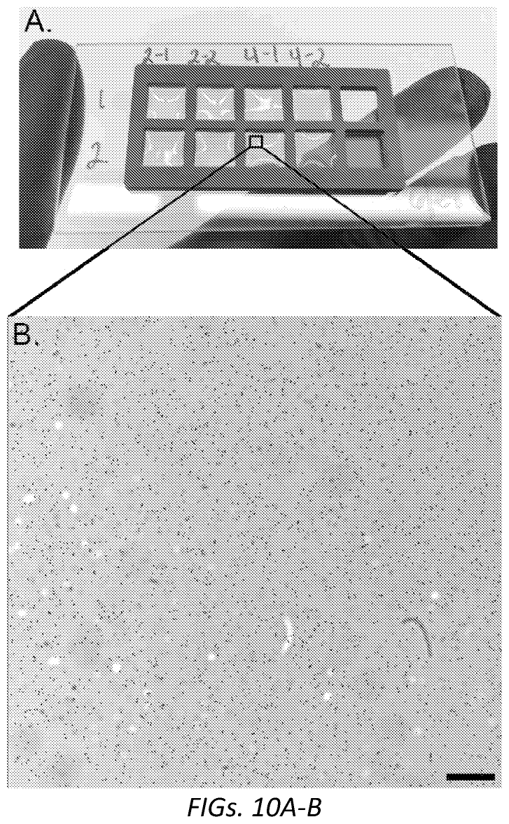

The second step of developing CARD-dELISA was to establish a method of bead immobilization for imaging and single molecule counting. Because the amplified enzymatic signal is already conjugated to the beads in the previous step of the assay, we were not restricted by the need to compartmentalize beads in microwells or droplets for enzymatic amplification. We used fibrin hydrogels for bead immobilization (FIG. 9B). Fibrin hydrogels are formed when thrombin enzymatically polymerizes fibrinogen to fibrin hydrogel networks.²⁸ Synthetic fibrin hydrogels are frequently used for applications including cell encapsulation and tissue engineering, and can easily be formed in situ.²⁹⁻³¹ Encapsulation of beads in a fibrin hydrogel is a fast and simple way to immobilize beads for imaging. FIG. 10A shows several bead samples encapsulated in fibrin hydrogels. To immobilize beads in the fibrin hydrogel, the bead solution is first drop cast onto a glass slide inside a silicon isolation well (7 mm×7 mm×2 mm). Solutions of fibrinogen and thrombin are mixed and immediately added to the isolation well. The hydrogel forms in ˜15 minutes and beads become trapped in the fibrin polymer network. A brightfield image of several hundred beads in the fibrin hydrogel is shown in FIG. 10B. This image is ˜100 μm × ˜100 μm, which represents a small region of the bead array. The entire bead array can be captured in −20-25 images at 10× magnification. Because the beads are relatively large (2.7 μm diameter) and quickly sediment out of solution, we observe that the beads are primarily in the same z-plane (i.e. at the interface of the glass slide and the fibrin hydrogel). In addition to being fast and simple, bead encapsulation in fibrin hydrogels is also a cheaper alternative compared to microwell arrays. This reduction in cost is important for developing a low-cost point-of-care digital ELISA platform.

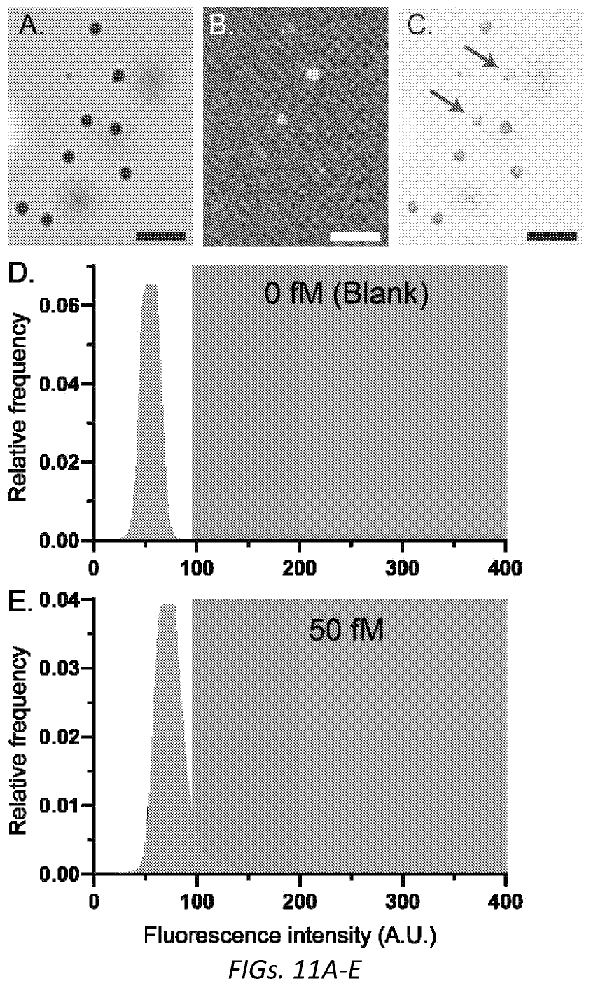

The final step of CARD-dELISA was to develop a method for single molecule counting. Brightfield and fluorescent images of the bead arrays are captured with an inverted fluorescence microscope and images are then analyzed using an algorithm in MATLAB to perform single molecule counting. Examples of a brightfield image (FIG. 11A) and a 488 nm fluorescence image (FIG. 11B) of a small region of interest are shown. Brightfield images are used to locate each bead, and the fluorescence images are used to identify beads with deposited tyramide-Alexa Fluor 488 dye from the signal amplification step. When the two images are overlaid (FIG. 11C), we observe that two of the eight beads in the images are “on” (grey arrows). The location of each bead is automatically determined by the MATLAB algorithm and the corresponding fluorescence intensity of each bead is calculated. For every sample, the fluorescence intensities of all beads are plotted as a histogram; a 0 fM IL-6 standard is plotted in FIG. 11D and a 50 fM IL-6 standard is plotted in FIG. 11E. The histogram for the blank sample in FIG. 11D is fitted to a normal distribution and the cutoff for “on” vs. “off” beads is set at four standard deviations above the mean fluorescence intensity of the blank (grey boxes in FIGS. 11D and 11E). AEB is calculated by dividing the number of “on” beads by the total number of beads. We analyze −50,000-60,000 beads per sample (i.e. −30% of beads are analyzed), which is an improvement in fraction of beads analyzed compared to conventional Simoa, where only −5% of beads are analyzed.

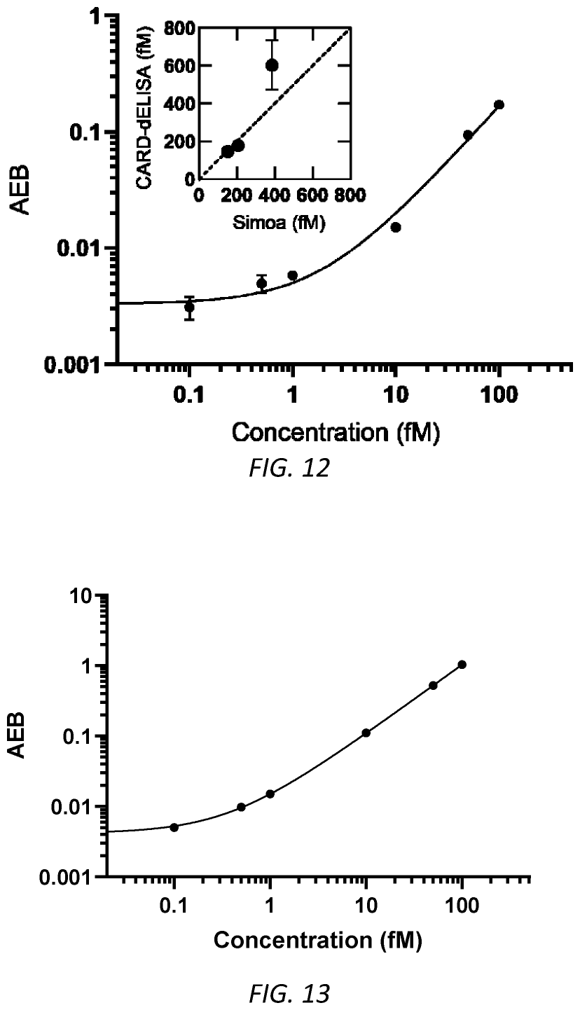

As a proof of concept, we used IL-6 as a model protein to generate a calibration curve using CARD-dELISA, which is plotted in FIG. 12; AEB values for the calibration curve are provided in Table 7. As expected, at low protein concentrations, most beads do not bind IL-6 (AEB values are small). As the IL-6 concentration increases, the number of beads that bind IL-6 increases. We also generated an IL-6 calibration curve (FIG. 13) using a standard Simoa assay on an HD-1 Analyzer. Both curves were fit with a four-parameter logistic (4PL) regression, which was used to estimate the LOD and LOQ of each assay (Table 8). CARD-dELISA yielded a wide dynamic range and an LOD of 1.36 fM for IL-6. Finally, we measured IL-6 in commercial saliva samples using CARD-dELISA and compared the results to conventional Simoa in order to confirm CARD-dELISA can be used to reliably detect proteins in biofluids. The results from both assays are reported in Table 9 and plotted in the inset of FIG. 12. We observe good agreement between the two methods (Spearman correlation coefficient is 1.00) demonstrating that CARD-dELISA can reliably detect IL-6 in saliva. Furthermore, like Simoa, CARD-dELISA allows for the use of highly diluted samples. Saliva samples analyzed by CARD-dELISA were diluted 25X. The use of high sample dilution factors reduces non-specific adsorption and therefore improves the accuracy of the assay. In addition, CARD-dELISA only required 10 μL of saliva, meaning protein biomarkers can be measured from small sample volumes.

TABLE 7
AEB values for IL-6 calibration curves for CARD-dELISA and
conventional Simoa

| | CARD-dELISA | | Simoa | |
|---|---|---|---|---|
| IL-6 Concentration | Average | | Average | |
| (fM) | AEB | CV (%) | AEB | CV (%) |
| 0 | 0.0030 | 30.1 | 0.0043 | 8.3 |
| 0.1 | 0.0031 | 22.1 | 0.0050 | 8.5 |
| 0.5 | 0.0050 | 17.1 | 0.0098 | 8.7 |
| 1 | 0.0058 | 3.5 | 0.0151 | 10.1 |
| 10 | 0.0151 | 7.5 | 0.1106 | 0.7 |
| 50 | 0.0937 | 4.2 | 0.5222 | 4.4 |
| 100 | 0.1708 | 1.6 | 1.0345 | 1.0 |
| 4PL fit R² | 0.943 | — | 0.999 | — |

TABLE 8
LOD and LOQ values for IL-6 calibration curves for CARD-
dELISA and conventional Simoa. LODs for CARD-dELISA and
Simoa were calculated as 3 standard deviations above background.
LOQs for CARD-dELISA and Simoa were calculated as 10
standard deviations above background. LOD for the Quanterix
commercial IL-6 assay was calculated as 2.5 standard deviations
above background

| | CARD- | | | CARD- | |
|---|---|---|---|---|---|
| | dELISA | Simoa | Quanterix | dELISA | Simoa |
| Marker | LOD (3×) | LOD (3×) | LOD (2.5×) | LOQ (10×) | LOD (10×) |
| IL-6 | 1.36 fM | 0.11 fM | 0.27 fM | 5.10 fM | 0.34 fM |

TABLE 9
Comparison of samples measured by CARD-dELISA and conventional
Simoa

| | CARD-dELISA | | Simoa | |
|---|---|---|---|---|
| | Mean | Standard | Mean | Standard |
| | Concentration | Deviation | Concentration | Deviation |
| Sample | (fM) | (fM) | (fM) | (fM) |
| 1 | 602 | 131 | 385 | 7.06 |
| 2 | 146 | 1.40 | 151 | 2.37 |
| 3 | 178 | 0.742 | 207 | 0.191 |

### REFERENCES FOR EXAMPLE 2

(1) Rissin, D. M.; et al. Single-Molecule Enzyme-Linked Immunosorbent Assay Detects Serum Proteins at Subfemtomolar Concentrations. Nat. Biotechnol. 2010, 28 (6), 595-599.
(2) Leirs, K.; et al. Bioassay Development for Ultrasensitive Detection of Influenza A Nucleoprotein Using Digital ELISA. Anal. Chem. 2016, 88 (17), 8450-8458.
(3) Cohen, L.; Walt, D. R. Highly Sensitive and Multiplexed Protein Measurements. Chem. Rev. 2019, 119 (1), 293-321.
(4) Song, L.; et al. Direct Detection of Bacterial Genomic DNA at Sub-Femtomolar Concentrations Using Single Molecule Arrays. Anal. Chem. 2013, 85 (3), 1932-1939.
(5) Cohen, L.; Hartman, M. R.; Amardey-Wellington, A.; Walt, D. R. Digital Direct Detection of MicroRNAs Using Single Molecule Arrays. Nucleic Acids Res. 2017, 45 (14), e137-e137.
(6) Wang, X.; Cohen, L.; Wang, J.; Walt, D. R. Competitive Immunoassays for the Detection of Small Molecules Using Single Molecule Arrays. J. Am. Chem. Soc. 2018, 140 (51), 18132-18139.
(7) Wang, X.; Walt, D. R. Simultaneous Detection of Small Molecules, Proteins and MicroRNAs Using Single Molecule Arrays. Chem. Sci. 2020.
(8) Rondelez, Y.; Tresset, G.; Tabata, K. V.; Arata, H.; Fujita, H.; Takeuchi, S.; Noji, H. Microfabricated Arrays of Femtoliter Chambers Allow Single Molecule Enzymology. Nat. Biotechnol. 2005, 23 (3), 361-365.
(9) Rissin, D. M.; Walt, D. R. Digital Concentration Readout of Single Enzyme Molecules Using Femtoliter Arrays and Poisson Statistics. Nano Lett. 2006, 6 (3), 520-523.
(10) Cohen, L.; Walt, D. R. Single-Molecule Arrays for Protein and Nucleic Acid Analysis. Annu. Rev. Anal. Chem. 2017, 10 (1), 345-363.
(11) Kim, S. H.; Iwai, S.; Araki, S.; Sakakihara, S.; Iino, R.; Noji, H. Large-Scale Femtoliter Droplet Array for Digital Counting of Single Biomolecules. Lab. Chip 2012, 12 (23), 4986.
(12) Witters, D.; Knez, K.; Ceyssens, F.; Puers, R.; Lammertyn, J. Digital Microfluidics-Enabled Single-Molecule Detection by Printing and Sealing Single Magnetic Beads in Femtoliter Droplets. Lab. Chip 2013, 13 (11), 2047.
(13) Yelleswarapu, V.; Buser, J. R.; Haber, M.; Baron, J.; Inapuri, E.; Issadore, D. Mobile Platform for Rapid Sub-Picogram-per-Milliliter, Multiplexed, Digital Droplet Detection of Proteins. Proc. Natl. Acad. Sci. 2019, 116 (10), 4489-4495.
(14) Cohen, L.; Cui, N.; Cai, Y.; Garden, P. M.; Li, X.; Weitz, D. A.; Walt, D. R. Single Molecule Protein Detection with Attomolar Sensitivity Using Droplet Digital Enzyme-Linked Immunosorbent Assay. ACS Nano 2020.
doi.org/10.1021/acsnano.0c02378.
(15) Wu, C.; Maley, A. M.; Walt, D. R. Single-Molecule Measurements in Microwells for Clinical Applications. Crit. Rev. Clin. Lab. Sci. 2020, 57 (4), 270-290.
(16) Mattsson, N.; Andreasson, U.; Zetterberg, H.; Blennow, K.; for the Alzheimer's Disease Neuroimaging Initiative. Association of Plasma Neurofilament Light With Neurodegeneration in Patients with Alzheimer Disease. JAMA Neurol. 2017, 74(5), 557.
(17) Shahim, P.; Tegner, Y.; Wilson, D. H.; Randall, J.; Skillback, T.; Pazooki, D.; Kallberg, B.; Blennow, K.; Zetterberg, H. Blood Biomarkers for Brain Injury in Concussed Professional Ice Hockey Players. JAMA Neurol. 2014, 71 (6), 684.
(18) Gill, J.; Latour, L.; Diaz-Arrastia, R.; Motamedi, V.; Turtzo, C.; Shahim, P.; Mondello, S.; DeVoto, C.; Veras, E.; Hanlon, D.; Song, L.; Jeromin, A. Glial Fibrillary Acidic Protein Elevations Relate to Neuroimaging Abnormalities after Mild TBI. Neurology 2018, 91 (15), e1385-e1389.
(19) Ng, A. S. L.; Tan, Y. J.; Lu, Z.; Ng, E. Y. L.; Ng, S. Y. E.; Chia, N. S. Y.; Setiawan, F.; Xu, Z.; Tay, K. Y.; Prakash, K. M.; Au, W. L.; Tan, E.; Tan, L. C. S. Plasma Alpha-synuclein Detected by Single Molecule Array Is Increased in PD. Ann. Clin. Transl. Neurol. 2019, 6 (3), 615-619.
(20) Wilson, D. H.; Hanlon, D. W.; Provuncher, G. K.; Chang, L.; Song, L.; Patel, P. P.; Ferrell, E. P.; Lepor, H.; Partin, A. W.; Chan, D. W.; Sokoll, L. J.; Cheli, C. D.; Thiel, R. P.; Fournier, D. R.; Duffy, D. C. Fifth-Generation Digital Immunoassay for Prostate-Specific Antigen by Single Molecule Array Technology. Clin. Chem. 2011, 57 (12), 1712-1721.
(21) Shi, Y.; Gao, W.; Lytle, N. K.; Huang, P.; Yuan, X.; Dann, A. M.; Ridinger-Saison, M.; DelGiorno, K. E.; Antal, C. E.; Liang, G.; Atkins, A. R.; Erikson, G.; Sun, H.; Meisenhelder, J.; Terenziani, E.; Woo, G.; Fang, L.; Santisakultarm, T. P.; Manor, U.; Xu, R.; Becerra, C. R.; Borazanci, E.; Von Hoff, D. D.; Grandgenett, P. M.; Hollingsworth, M. A.; Leblanc, M.; Umetsu, S. E.; Collisson, E. A.; Scadeng, M.; Lowy, A. M.; Donahue, T. R.; Reya, T.; Downes, M.; Evans, R. M.; Wahl, G. M.; Pawson, T.; Tian, R.; Hunter, T. Targeting LIF-Mediated Paracrine Interaction for Pancreatic Cancer Therapy and Monitoring. Nature 2019, 569 (7754), 131-135.
(22) Olsen, D. A.; Kjaer, I. M.; Brandslund, I. Development of a Three-Plex Single Molecule Immunoassay Enabling Measurement of the EGFR Ligands Amphiregulin, Betacellulin and Transforming Growth Factor α Simultaneously in Human Serum Samples. J. Immunol. Methods 2018, 459, 63-69.
(23) Banz, A.; Lantz, A.; Riou, B.; Foussadier, A.; Miller, M.; Davies, K.; Wilcox, M. Sensitivity of Single-Molecule Array Assays for Detection of Clostridium Difficile Toxins in Comparison to Conventional Laboratory Testing Algorithms. J. Clin. Microbiol. 2018, 56 (8).
(24) Anderson, A. M.; Tyor, W. R.; Mulligan, M. J.; Waldrop-Valverde, D.; Lennox, J. L.; Letendre, S. L. Measurement of Human Immunodeficiency Virus P24 Antigen in Human Cerebrospinal Fluid With Digital Enzyme-Linked Immunosorbent Assay and Association With Decreased Neuropsychological Performance. Clin. Infect. Dis. 2018, 67 (1), 137-140.
(25) Ahmad, R.; Xie, L.; Pyle, M.; Suarez, M. F.; Broger, T.; Steinberg, D.; Ame, S. M.; Lucero, M. G.; Szucs, M. J.; MacMullan, M.; Berven, F. S.; Dutta, A.; Sanvictores, D. M.; Tallo, V. L.; Bencher, R.; Eisinger, D. P.; Dhingra, U.; Deb, S.; Ali, S. M.; Mehta, S.; Fawzi, W. W.; Riley, I. D.; Sazawal, S.; Premji, Z.; Black, R.; Murray, C. J. L.; Rodriguez, B.; Carr, S. A.; Walt, D. R.; Gillette, M. A. A Rapid Triage Test for Active Pulmonary Tuberculosis in Adult Patients with Persistent Cough. Sci. Transl. Med. 2019, 11 (515), eaaw8287.
(26) Kosack, C. S.; Page, A.-L.; Klatser, P. R. A Guide to Aid the Selection of Diagnostic Tests. Bull. World Health Organ. 2017, 95 (9), 639-645.
(27) Akama, K.; Shirai, K.; Suzuki, S. Droplet-Free Digital Enzyme-Linked Immunosorbent Assay Based on a Tyramide Signal Amplification System. Anal. Chem. 2016, 88 (14), 7123-7129.
(28) Doolittle, R. F. FIBRINOGEN AND FIBRIN. Annu. Rev. Biochem. 53, 195-229.
(29) Calderon, G. A.; Thai, P.; Hsu, C. W.; Grigoryan, B.; Gibson, S. M.; Dickinson, M. E.; Miller, J. S. Tubulogenesis of Co-Cultured Human IPS-Derived Endothelial Cells and Human Mesenchymal Stem Cells in Fibrin and Gelatin Methacrylate Gels. Biomater. Sci. 2017, 5 (8), 1652-1660.
(30) Black, L. D.; Meyers, J. D.; Weinbaum, J. S.; Shvelidze, Y. A.; Tranquillo, R. T. Cell-Induced Alignment Augments Twitch Force in Fibrin Gel-Based Engineered Myocardium via Gap Junction Modification. Tissue Eng. Part A 2009, 15 (10), 3099-3108.
(31) Syedain, Z. H.; Weinberg, J. S.; Tranquillo, R. T. Cyclic Distension of Fibrin-Based Tissue Constructs: Evidence of Adaptation during Growth of Engineered Connective Tissue. Proc. Natl. Acad. Sci. 2008, 105 (18), 6537-6542.
(32) Liu, D.; Li, X.; Zhou, J.; Liu, S.; Tian, T.; Song, Y.; Zhu, Z.; Zhou, L.; Ji, T.; Yang, C. A Fully Integrated Distance Readout ELISA-Chip for Point-of-Care Testing with Sample-in-Answer-out Capability. Biosens. Bioelectron. 2017, 96, 332-338.
(33) Berry, S. M.; Alarid, E. T.; Beebe, D. J. One-Step Purification of Nucleic Acid for Gene Expression Analysis via Immiscible Filtration Assisted by Surface Tension (IFAST). Lab. Chip 2011, 11 (10), 1747.
(34) Wei, Q.; Qi, H.; Luo, W.; Tseng, D.; Ki, S. J.; Wan, Z.; Gorocs, Z.; Bentolila, L. A.; Wu, T.-T.; Sun, R.; Ozcan, A. Fluorescent Imaging of Single Nanoparticles and Viruses on a Smart Phone. ACS Nano 2013, 7 (10), 9147-9155.
(35) Preechaburana, P.; Suska, A.; Filippini, D. Biosensing with Cell Phones. Trends Biotechnol. 2014, 32 (7), 351-355.
(36) Li, Z.; Zhang, S.; Yu, T.; Dai, Z.; Wei, Q. Aptamer-Based Fluorescent Sensor Array for Multiplexed Detection of Cyanotoxins on a Smartphone. Anal. Chem. 2019, 91 (16), 10448-10457.
(37) Novak, R.; et al. Robotic Fluidic Coupling and Interrogation of Multiple Vascularized Organ Chips. Nat. Biomed. Eng. 2020, 4 (4), 407-420.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of detecting a biomolecule in a sample, the method comprising:
providing a solution comprising the sample;
contacting the solution with a plurality of beads comprising a capture moiety that binds to the biomolecule, under conditions and for a time sufficient for biomolecules in the sample to bind to the capture moiety;
contacting the solution with a binding moiety that binds to the biomolecule and allows for generation of an on-bead non-diffusible detectable signal sufficient to allow detection of each bead carrying a target molecule, and then generating the amplified signal;
immobilizing the beads, optionally in a monolayer; and
detecting the signal.

2. The method of claim 1, wherein immobilizing the beads comprises dropcasting the solution comprising the beads onto a slide, or catalyzing gelation of the solution.

3. The method of claim 1, further comprising contacting the solution with a signal amplification moiety that binds to the binding moiety.

4. The method of claim 3, wherein the signal amplification moiety comprises an enzyme or branched DNA.

5. The method of claim 1, wherein detecting the signal comprises imaging the beads to detect a fluorescent or other signal.

6. The method of claim 1, further comprising determining a number and/or percentage of beads that comprise bead-biomolecule complexes.

7. The method of claim 1, wherein the bead comprises a polymer, metal, metal-oxide, semiconductor, and/or semiconductor oxide.

8. The method of claim 1, wherein the detectable signal is generated by rolling circle amplification followed by hybridization with a complementary fluorescently labeled DNA probe; Tyramide Signal Amplification (TSA); hybridization chain reaction; Enzyme-catalyzed proximity labeling (PL) polymerization; Polymerization-based signal amplification; or Magnetic Bead—Quantum Dot Immunoassays.

9. The method of claim 1, wherein the detectable signal is generated by a pre-amplified signal.

10. The method of claim 8, wherein the pre-amplified signal is a labeled polymer or nanoparticle.

11. The method of claim 1, wherein the beads are dropcast onto a surface and allowed to dry before the signal is detected.

12. The method of claim 1, wherein the solution is applied to, or in contact with, a surface and gelation is catalyzed before the signal is detected.

13. The method of claim 11, wherein the surface is a slide, chip, or flowcell.

14. The method of claim 1, wherein catalyzing gelation of the solution comprises mixing fibrinogen and/or thrombin; fibrin; cellulose; collagen; gelatin; agarose; hyaluronic acid; polyhydroxyethylmethacrylate (poly(HEMA)); polyethylene glycol (PEG); or acrylamide into the solution.

15. The method of claim 1, wherein the solution comprises a polymer selected from fibrinogen and/or thrombin; fibrin; cellulose; collagen; gelatin; agarose; hyaluronic acid; polyhydroxyethylmethacrylate (poly(HEMA)); polyethylene glycol (PEG); or acrylamide; and the method comprises catalyzing gelation of into polymer.


SEQUENCE LISTING (3 sequences)

SEQ ID NO: 1 - DNA, 39 bp, Artificial Sequence
  Primer sequence
ttttttttttttttttagacaccgttccttggacagagc

SEQ ID NO: 2 - DNA, 80 bp, Artificial Sequence
  Template sequence
gaacggtgtctattatgtcctatcctcagctattatgtcctatcctcagctattatgtcctatcctcagctctgtccaag

SEQ ID NO: 3 - DNA, 20 bp, Artificial Sequence
  Probe sequence
tattatgtcctatcctcagc